(19) United States
(12) Patent Application Publication
(10) Pub. No.: US 2010/0240746 A1
(43) Pub. Date: Sep. 23, 2010

(54) STEREOISOMERS OF TRICYCLODECAN-9-YL-XANTHOGENATE

(75) Inventors: Miyuki Tomioka, Basel (CH); Ko Hasegawa, Tokyo (JP)

Correspondence Address: JONES DAY, 222 EAST 41ST ST, NEW YORK, NY, 10017, US

(73) Assignee: LUMAVITA AG, Basel (CH)

(21) Appl. No.: 12/666,282

(22) PCT Filed: Jul. 3, 2008
(86) PCT No.: PCT/EP2008/005460

§ 371 Date: Jun. 4, 2010

Related U.S. Application Data
(60) Provisional application No. 60/958,370, filed on Jul. 3, 2007.

(51) Int. Cl. A61K 31/265; C07C 329/14; A61P 31/12; A61P 35/00; A61P 29/00; A61P 25/00; C12P 15/00

(52) U.S. Cl. 514/511; 558/247; 435/127

(57) ABSTRACT

Provided herein are optically active stereoisomers of tricylclodecan-9-yl xanthogenate, processes of preparation, and pharmaceutical compositions thereof. Also provided are methods of their use for treating, preventing, or ameliorating one or more symptoms of a disease caused by a virus.

### CROSS REFERENCE TO RELATED APPLICATION

This application claims the benefit of the priority of U.S. Provisional Application No. 60/958,370, filed Jul. 3, 2007, the disclosure of which is incorporated herein by reference in its entirety.

### FIELD

Provided herein are optically active stereoisomers of tricylclodecan-9-yl xanthogenate, processes of preparation, and pharmaceutical compositions thereof. Also provided are methods of their use for treating, preventing, or ameliorating viral infections and diseases caused by such infections.

### BACKGROUND

Tricyclodecan-9-yl-xanthogenate is a complex molecule that contains five chiral centers, which may lead to 32 theoretical stereoisomers. Due to its constrained ring structure, however, the molecule exists in fewer stereoisomers than the theoretical possibility. Some stereoisomers are shown in Scheme 1 below, including four enantiomeric pairs, O-exo/C-exo, (9R)-1A and (9S)-1A; O-exo/C-endo, (9R)-1B and (9S)-1B; O-endo/C-exo, (9R)-1C and (9S)-1C; and O-endo/C-endo, (9R)-1D and (9S)-1D.

British Patent GB 2,091,244; and U.S. Pat. Nos. 4,602,037 and 4,981,869 describe a mixture of stereoisomers of tricyclodecan-9-yl-xanthogenate, which is known as D609. As characterized in U.S. Application Publication No. 2005/0085448, D609 contains 83% of racemic O-exo/C-exo stereoisomers 1A and 17% of racemic O-exo/C-endo 1B, O-endo/C-exo 1C, and O-endo/C-endo 1D.

D609 has been reported to exhibit a variety of biological activity, including antitumor (U.S. Pat. No. 4,602,037; Amtmann and Sauer, Cancer Lett. 1987, 35, 237-244; Furstenberger et al., Int. J. Cancer 1989, 43, 508-512; Schick et al., Cancer Lett. 1989, 46, 143-147; Schick et al., Cancer Lett. 1989, 46, 149-152; Sauer et al., Cancer Lett. 1990, 53, 97-102; Porn-Ares et al., Exp. Cell. Res. 1997, 235, 48-54), antiviral (Sauer et al., Pro. Natl. Acad. Sci. USA 1984, 81, 3263-3267; Amtmann et al., Biochem. Pharmacol. 1987, 36, 1545-1549; Villanueva et al., Virology 1991, 181, 101-108; Walro and Rosenthal, Antiviral Res. 1997, 36, 63-72), and anti-inflammatory activity (Machleidt et al., J. Exp. Med. 1996, 184, 725-733; Tschaikowsky et al., J. Pharmacol. 1998, 285, 800-804).

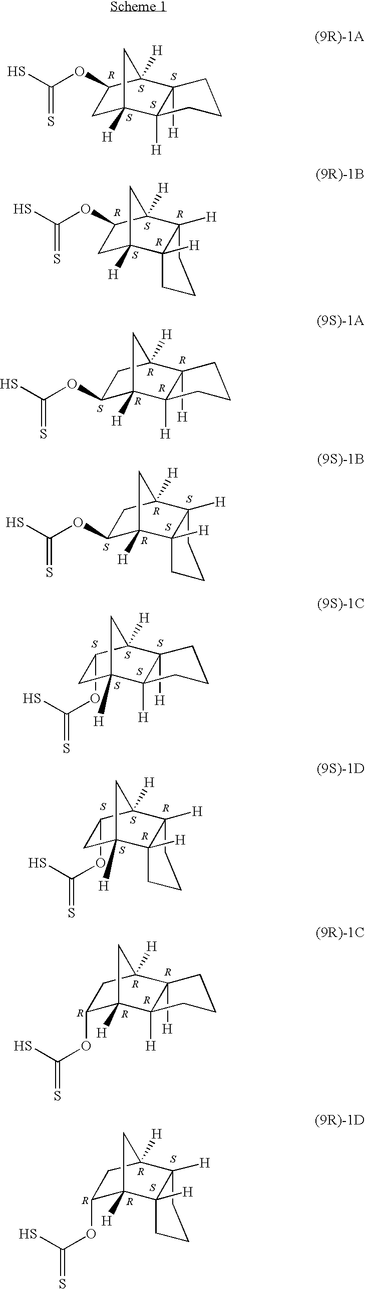

D609 has also been reported as a specific inhibitor of phosphatidylcholine-specific phospholipase C(PC-PLC) (Amtmann, Drugs Exp. Clin. Res. 1996, 22, 287-294; Muller-Decker, Biochem. Biophys. Res. Commun. 1989, 162, 198-205). Hydrolysis of phosphatidylcholine by PC-PLC generates a second messenger, diacylglycerol, which activates protein kinase C (PKC) and/or acidic sphingomyelinase (aSMase). Inhibition of PC-PLC by D609 has been suggested to be useful to suppress the activities of PKC and aSMase (Schutze et al., Cell 1992, 71, 765-776; Wiegmann et al., Cell 1994, 78, 1005-1015; Cifone et al., EMBO J. 1995, 14, 5859-5868; Amtmann, Drugs Exp. Clin. Res. 1996, 22, 287-294; Machleidt et al., J. Exp. Med. 1996, 184, 725-733; Yamamoto et al., Biochem. J. 1997, 325, 223-228). Suppression of PKC may partly account for the antiproliferative and antitumor activity of D609 (Muller-Decker et al., Exp. Cell Res. 1988, 177, 295-302; Muller-Decker et al., Biochem. Biophys. Res. Commun. 1989, 162, 198-205; Amtmann, Drugs Exp. Clin. Res. 1996, 22, 287-294). Suppression of aSMase by D609 may lead to the reduction in ceramide production, thus inhibiting ceramide-mediated signal transduction (Schutze et al., Cell 1992, 71, 765-776; Wiegmann et al., Cell 1994, 78, 1005-1015; Machleidt et al., J. Exp. Med. 1996, 184, 725-733), such as activation of PKC-z (Simarro et al., J. Immunol. 1999, 162, 5149-5155), mitogen-activated protein kinase (Buscher et al., Mol. Cell. Biol. 1995, 15, 466-475; Monick et al., J. Immunol. 1999, 162, 3005-3012), and nuclear factor-kB (NF-kB) (Cell 1992, 71, 765-776; Wiegmann et al., Cell 1994, 78, 1005-1015).

Additionally, U.S. Pat. No. 4,851,435; WO 96/14841; and U.S. Application Publication Nos. 2004/0122086 and 2005/0085448 describe the use of adjuvants, such as ionic detergent, lipids, and steroids, to enhance the therapeutic efficacy of D609 as an antiviral or antitumor agent.

Gonzalez-Roura et al. (Lipid 2002, 37, 401-406) and U.S. Patent Application Publication No. 2005/0085448 describe the synthesis of racemic O-exo/C-exo 1A, O-exo/C-endo 1B, O-endo/C-exo 1C, and O-endo/C-endo 1D stereoisomers. However, Gonzalez-Roura et al. reported no significant differences between these diastereomers in their inhibitory activities against PC-specific phospholipase C.

The above-mentioned biological studies were conducted using D609, a complex distereomeric mixture, or racemic mixtures of tricylclodecan-9-yl xanthogenate. It would be particularly desirable to find an optically pure stereoisomer of tricylclodecan-9-yl xanthogenate having a compound having the therapeutic advantages of D609, but which avoids or reduces unintended, undesired, unwanted, adverse or side effects of D609 or other antivirals.

Citation of any reference herein is not an admission that such reference is prior art to the present application.

### SUMMARY OF THE DISCLOSURE

Provided herein is optically active (−)-O-exo/C-exo-tricyclo[5.2.1.0^{2,6}]-dec-9-yl-xanthic acid, or a pharmaceutically acceptable salt, solvate, or prodrug thereof. In one embodiment, provided herein is a pharmaceutically acceptable salt of the optically active (−)-O-exo/C-exo-tricyclo[5.2.1.0^{2,6}]-dec-9-yl-xanthic acid, including, but not limited to, lithium, magnesium, calcium, sodium, potassium, and zinc salt.

This optically active single stereoisomer has utility in pharmaceutical compositions and methods for treating viral infection. To the knowledge of the inventors, no report exists of the synthesis or separation of a single enantiomer from many stereoisomers of tricylclodecan-9-yl xanthogenate or from the racemic O-exo/C-exo-tricyclo[5.2.1.0^{2,6}]-dec-9-yl-xanthic acid or derivatives thereof. Provided herein are three methods for making such optically active enantiomer.

In one embodiment, the optically active (−)-O-exo/C-exo-tricyclo[5.2.1.0^{2,6}]-dec-9-yl-xanthic acid, or a pharmaceutically acceptable salt, solvate, or prodrug thereof, is synthesized through asymmetric hydrosilylation of an alkene 5:

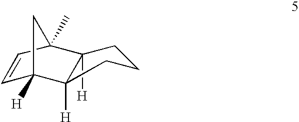

in the presence of a transition metal catalyst complexed with a chiral monodentate phosphine.

In another embodiment, the optically active (−)-O-exo/C-exo-tricyclo[5.2.1.0^{2,6}]-dec-9-yl-xanthic acid, or a pharmaceutically acceptable salt, solvate, or prodrug thereof, is produced through enzymatic resolution of a racemic mixture of a unsaturated ester 11:

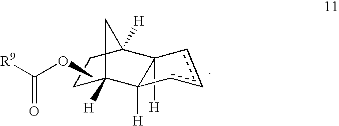

In still another embodiment, the optically active (−)-O-exo/C-exo-tricyclo[5.2.1.0^{2,6}]-dec-9-yl-xanthic acid, or a pharmaceutically acceptable salt, solvate, or prodrug thereof, is prepared through enzymatic resolution of a racemic mixture of a saturated ester 13:

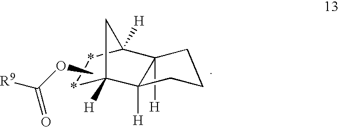

Also provided herein are pharmaceutical compositions comprising optically active (−)-O-exo/C-exo-tricyclo[5.2.1.0^{2,6}]-dec-9-yl-xanthic acid, or a pharmaceutically acceptable salt, solvate, or prodrug thereof; in combination with one or more pharmaceutically acceptable excipients or carriers. In certain embodiments, the pharmaceutical compositions comprise a pharmaceutically acceptable salt of optically active (−)-O-exo/C-exo-tricyclo[5.2.1.0^{2,6}]-dec-9-yl-xanthic acid, such as lithium, magnesium, calcium, sodium, potassium, or zinc salt. In certain embodiments, the pharmaceutical compositions are provided as a dosage form for topical administration.

Further provided herein is a method for treating, preventing, or ameliorating one or more symptoms of a disease caused by a virus, which comprises administering to a subject a therapeutically effective amount of optically active (−)-O-exo/C-exo-tricyclo[5.2.1.0^{2,6}]-dec-9-yl-xanthic acid, or a pharmaceutically acceptable salt, solvate, or prodrug thereof. In one embodiment, the disease is a sexually transmitted disease. In another embodiment, the virus is an oncogenic virus. In yet another embodiment, the virus is pallipoma virus. In still another embodiment, the virus is herpes simplex virus.

Provided herein is a method for inhibiting the replication of a virus, which comprises contacting the virus with an effective amount of optically active (−)-O-exo/C-exo-tricyclo[5.2.1.0^{2,6}]-dec-9-yl-xanthic acid, or a pharmaceutically acceptable salt, solvate, or prodrug thereof. In one embodiment, the virus is a sexually transmissible. In another embodiment, the virus is an oncogenic virus. In yet another embodiment, the virus is pallipoma virus. In still another embodiment, the virus is herpes simplex virus.

Provided herein is a method for inhibiting the activity of phosphatidylcholine-specific phospholipase C, which comprises contacting the phospholipase C with optically active (−)-O-exo/C-exo-tricyclo[5.2.1.0^{2,6}]-dec-9-yl-xanthic acid, or a pharmaceutically acceptable salt, solvate, or prodrug thereof.

### BRIEF DESCRIPTION OF THE FIGURES

FIG. 1 illustrates the effect of optically active (−)-O-exo/C-exo-tricyclo[5.2.1.0^{2,6}]-dec-9-yl-xanthic acid, or a pharmaceutical acceptable salt thereof, on the growth of HPV-31-infected CIN612 9E keratinocytes in a single passage, in comparison with the effect of INF-γ.

FIG. 2 illustrates the effect of optically active (−)-O-exo/C-exo-tricyclo[5.2.1.0^{2,6}]-dec-9-yl-xanthic acid, or a pharmaceutical acceptable salt thereof, on HPV-31-specific RNA and DNA levels, and cell proliferation in HPV-31-infected CIN612 9E keratinocytes.

FIG. 3 illustrates the effect of optically active (−)-O-exo/C-exo-tricyclo[5.2.1.0^{2,6}]-dec-9-yl-xanthic acid, or a pharmaceutical acceptable salt thereof, on the growth of HPV-31-infected CIN612 9E keratinocytes in multiple passage, in comparison with INF-γ.

FIG. 4 illustrates the effect of optically active (−)-O-exo/C-exo-tricyclo[5.2.1.0^{2,6}]-dec-9-yl-xanthic acid, or a pharmaceutical acceptable salt thereof, on the growth of A431 cells in multiple passages, in comparison with INF-γ.

FIG. 5 illustrate cell morphology of (A) HPV-31-infected CIN612 9E keratinocytes being treated with optically active (−)-O-exo/C-exo-tricyclo[5.2.1.0^{2,6}]-dec-9-yl-xanthic acid or a pharmaceutical acceptable salt thereof (A); and (B) untreated CIN612 9E cells.

FIG. 6 illustrates the effect of optically active (−)-O-exo/C-exo-tricyclo[5.2.1.0^{2,6}]-dec-9-yl-xanthic acid, or a pharmaceutical acceptable salt thereof, on HPV-31-specific DNA level in HPV-31-infected CIN612 9E keratinocytes in multiple passages, in comparison with INF-γ.

FIG. 7 illustrates the effect of optically active (−)-O-exo/C-exo-tricyclo[5.2.1.0^{2,6}]-dec-9-yl-xanthic acid, or a pharmaceutical acceptable salt thereof, on HPV-31-specific RNA level in HPV-31-infected CIN612 9E keratinocytes in multiple passages, in comparison with INF-γ.

### DETAILED DESCRIPTION

To facilitate understanding of the disclosure set forth herein, a number of terms are defined below.

As used herein, the singular forms “a,” “an,” and “the” may refer to plural articles unless specifically stated otherwise. Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The terms “prevent,” “preventing,” and “prevention” refer to a method of delaying or precluding the onset of a disorder, disease, or condition; and/or one or more of the symptoms associated with the disorder, disease, or condition; barring a subject from acquiring a disease or reducing a subject's risk of acquiring a disorder, disease, or condition.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, Fla., 2004.

The terms “active ingredient” and “active substance” refer to a compound, which is administered, alone or in combination with one or more pharmaceutically acceptable excipients, to a subject for treating, preventing, or ameliorating one or more symptoms of a disorder or disease. As used herein, “active ingredient” and “active substance” specifically refer an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a disorder or disease.

The term “release controlling excipient” refers to an excipient whose primary function is to modify the duration or place of release of an active substance from a dosage form as compared with a conventional immediate release dosage form.

The term “nonrelease controlling excipient” refers to an excipient whose primary function do not include modifying the duration or place of release of an active substance from a dosage form as compared with a conventional immediate release dosage form.

The term “alkyl” refers to a linear saturated monovalent hydrocarbon radical or a branched saturated monovalent hydrocarbon radical. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl may be substituted with one or more substituents Q as described herein.

The term “cycloalkyl” refers to a cyclic saturated bridged or non-bridged monovalent hydrocarbon radical, which may be optionally substituted one or more substituents Q as described herein. In certain embodiments, the cyclalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, decalinyl, and adamantyl.

The term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may also be optionally substituted with one or more substituents Q as described herein.

The term “heteroaryl” refers to a monocyclic aromatic group and/or multicyclic aromatic group that contains at least one aromatic ring, wherein at least one aromatic ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. The heteroaryl may be attached to the main structure at any heteroatom or carbon atom which results in the creation of a stable compound. In certain embodiments, the heteroaryl has from 5 to 20, from 5 to 15, or from 5 to 10 ring atoms. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. In certain embodiments, heteroaryl may also be optionally substituted with one or more substituents Q as described herein.

The term “heterocyclyl” or “heterocyclic” refers to a monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may includes a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to: acridinyl, azepinyl, benzimidazolyl, benzindolyl, benzoisoxazolyl, benzisoxazinyl, benzo[4,6]imidazo[1,2 a]pyridinyl, benzodioxanyl, benzodioxolyl, benzofuranonyl, benzofuranyl, benzonaphthofuranyl, benzopyranonyl, benzopyranyl, benzotetrahydrofuranyl, benzotetrahydrothienyl, benzothiadiazolyl, benzothiazolyl, benzothiophenyl, benzotriazolyl, benzothiopyranyl, benzoxazinyl, benzoxazolyl, benzothiazolyl, β carbolinyl, carbazolyl, chromanyl, chromonyl, cinnolinyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4 dithianyl, furanonyl, furanyl, imidazolidinyl, imidazolinyl, imidazolyl, imidazopyridinyl, imidazothiazolyl, indazolyl, indolinyl, indolizinyl, indolyl, isobenzotetrahydrofuranyl, isobenzotetrahydrothienyl, isobenzothienyl, isochromanyl, isocoumarinyl, isoindolinyl, isoindolyl, isoquinolinyl, isothiazolidinyl, isothiazolyl, isoxazolidinyl, isoxazolyl, morpholinyl, naphthyridinyl, octahydroindolyl, octahydroisoindolyl, oxadiazolyl, oxazolidinonyl, oxazolidinyl, oxazolopyridinyl, oxazolyl, oxiranyl, perimidinyl, phenanthridinyl, phenathrolinyl, phenarsazinyl, phenazinyl, phenothiazinyl, phenoxazinyl, phthalazinyl, piperazinyl, piperidinyl, 4 piperidonyl, pteridinyl, purinyl, pyrazinyl, pyrazolidinyl, pyrazolyl, pyridazinyl, pyridinyl, pyridopyridinyl, pyrimidinyl, pyrrolidinyl, pyrrolinyl, pyrrolyl, quinazolinyl, quinolinyl, quinoxalinyl, quinuclidinyl, tetrahydrofuryl, tetrahydrofuranyl, tetrahydroisoquinolinyl, tetrahydropyranyl, tetrahydrothienyl, tetrazolyl, thiadiazolopyrimidinyl, thiadiazolyl, thiamorpholinyl, thiazolidinyl, thiazolyl, thienyl, triazinyl, triazolyl and 1,3,5 trithianyl. In certain embodiments, heterocyclic may also be optionally substituted with one or more substituents Q as described herein.

The term “acyl” refers to a —C(O)R radical, wherein R is alkyl, cycloalkyl, alkenyl, heterocyclyl, aryl, or heteroaryl, each as defined herein. Examples of acyl groups include, but are not limited to, acetyl, propionyl, butanoyl, isobutanoyl, pentanoyl, hexanoyl, heptanoyl, octanoyl, nonanoyl, decanoyl, dodecanoyl, tetradecanoyl, hexadecanoyl, octadecanoyl, eicosanoyl, docosanoyl, myristoleoyl, palmitoleoyl, oleoyl, linoleoyl, arachidonoyl, benzoyl, pyridinylcarbonyl, and furoyl.

The term “halogen”, “halide” or “halo” refers to fluorine, chlorine, bromine, or iodine.

The term “optionally substituted” is intended to mean that a group, such as an alkyl, alkylene, alkenyl, alkynyl, cycloalkyl, aryl, heteroaryl, or heterocyclyl group, may be substituted with one or more substituents independently selected from, e.g., halo, cyano (—CN), nitro (—NO₂), —SR^{a}, —S(O)R^{a}, —S(O)₂R^{a}, —R^{a}, —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{C}, —OC(═NR^{a})NR^{b}R^{c}, —OS(O)R^{a}, —OS(O)₂R^{a}, —OS(O)NR^{a}R^{b}, —OS(O)₂ NR^{a}R^{b}, —NR^{a}R^{b}, —NR^{a}C(O)R^{b}, —NR^{a}C(O)OR^{b}, —NR^{a}C(O)NR^{b}R^{c}, —NR^{a}C(NR^{b})NR^{c}R^{d}, —NR^{a}S(O)R^{b}, —NR^{a}S(O)₂R^{b}, —NR^{a}S(O)R^{b}R^{c}, or —NR^{a}S(O)₂R^{b}R^{c}; wherein R^{a}, R^{b}, R^{c}, and R^{d} are each independently, e.g., alkyl, alkenyl, cycloalkyl, aryl, heteroaryl, or heterocyclyl.

The term “optionally substituted” is intended to mean that a group, such as alkyl, cycloalkyl, aryl, heteroaryl, heterocyclyl, or acyl, may be substituted with one or more substituents Q, in one embodiment, one, two, three, four substituents Q, where each Q is independently selected from the group consisting of cyano, halo, and nitro; C₁₋₆ alkyl, C₂₋₆ alkenyl, C₂₋₆ alkynyl, C₃₋₇ cycloalkyl, C₆₋₁₄ aryl, heteroaryl, and heterocyclyl; and —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, and —S(O)₂R^{e}; wherein each R^{e}, R^{f}, R^{g}, and R^{h} is independently hydrogen; C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} together with the N atom to which they are attached form heterocyclyl.

The terms “optically active” and “enantiomerically active” are used herein interchangeably, and refer to a compound comprising at least a sufficient excess of one enantiomer over the other such that the compound mixture rotates plane polarized light. The optical activity of an enantiomer is typically expressed as enantiomeric excess (e.e.). In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, or no less than about 94% no less than about 95%, no less than about 96%, no less than about 97%, no less than about 98%, no less than about 99%, or no less than about 99.5%, no less than about 99.8%. In certain embodiments, the compound comprises about 95% or more of the (−) enantiomer and about 5% or less of the (+) enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrarotatory, that is, the compound rotates the plane of polarized light to the right or clockwise. However, the sign of optical rotation, (+) and (−), is not related to the absolute configuration of the molecule, R and S.

The term “solvate” refers to a compound provided herein or a salt thereof, which further includes a stoichiometric or non-stoichiometric amount of solvent bound by non-covalent intermolecular forces. Where the solvent is water, the solvate is a hydrate.

The term “IC₅₀” refers an amount, concentration, or dosage of a compound that is required for 50% inhibition of a maximal response in an assay that measures such response.

The terms “tricylclodecan-9-yl xanthogenate” refers to tricyclo[5.2.1.0^{2,6}]-dec-9-yl-xanthic acid of Formula 1, or a pharmaceutically acceptable salt or solvate.

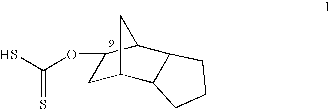

### Tricyclo[5.2.1.0^{2,6}]-dec-9-yl-xanthic Acid

Tricyclo[5.2.1.0^{2,6}]-dec-9-yl-xanthic acid is a complex molecule that contains five chiral centers, which may lead to 32 theoretical stereoisomers. Due to its constrained ring structure, however, the molecule exists in fewer stereoisomers than the theoretical possibility. Some stereoisomers are shown in Scheme 1, including four enantiomeric pairs, O-exo/C-exo, (9R)-1A and (9S)-1A; O-exo/C-endo, (9R)-1B and (9S)-1B; O-endo/C-exo, (9R)-1C and (9S)-1C; and O-endo/C-endo, (9R)-1D and (9S)-1D.

This disclosure contemplates several advantages of employing optically active (−)-O-exo/C-exo-tricyclo[5.2.1.0^{2,6}]-dec-9-yl-xanthic acid 1A over a racemic or diastereomeric mixture as a therapeutic agent. First, the optically pure (−)-O-exo/C-exo-tricyclo[5.2.1.0^{2,6}]-dec-9-yl-xanthic acid 1A is significantly potent, thus permitting the use of lower dose or concentration to achieve the same benefit in comparison with either a racemic or diastereomeric mixture. Second, the disclosure contemplates reducing or avoiding unwanted, undesired, adverse or side effects associated with the use of a diastereomeric or racemic mixture. In fact, the optically pure (−)-O-exo/C-exo-tricyclo[5.2.1.0^{2,6}]-dec-9-yl-xanthic acid 1A has an enhanced therapeutic index in comparison with either a racemic or diastereomeric mixture (see, Examples as disclosed herein). Other benefits contemplated by the disclosure of using the optically pure isomer described herein or composition thereof, may include simplification of pharmacokinetic profile, reduction of undesirable drug-drug interactions, and reduction of patient-to-patient variations.

Accordingly, provided herein is optically active (−)-O-exo/C-exo-tricyclo[5.2.1.0^{2,6}]-dec-9-yl-xanthic acid 1A, or a pharmaceutically acceptable salt, solvate, or prodrug thereof.

In certain embodiments, the optically active (−)-O-exo/C-exo-tricyclo[5.2.1.0^{2,6}]-dec-9-yl-xanthic acid 1A, or a pharmaceutically acceptable salt, solvate, or prodrug thereof, has an enantiomeric excess of no less than about 50%, no less than about 60%, no less than about 70%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, no less than about 99.9%, no less than about 99.95%, no less than about 99.99%, or about 100%.

In certain embodiments, the enantiomeric excess of the optically active (−)-O-exo/C-exo-tricyclo[5.2.1.0^{2,6}]-dec-9-yl-xanthic acid 1A, or a pharmaceutically acceptable salt, solvate, or prodrug thereof, is no less than about 50%. In certain embodiments, the enantiomeric excess is no less than about 60%. In certain embodiments, the enantiomeric excess is no less than about 70%. In certain embodiments, the enantiomeric excess is no less than about 80%. In certain embodiments, the enantiomeric excess is no less than about 85%. In certain embodiments, the enantiomeric excess is no less than about 90%. In certain embodiments, the enantiomeric excess is no less than about 91%. In certain embodiments, the enantiomeric excess is no less than about 92%. In certain embodiments, the enantiomeric excess is no less than about 93%. In certain embodiments, the enantiomeric excess is no less than about 94%. In certain embodiments, the enantiomeric excess is no less than about 95%. In certain embodiments, the enantiomeric excess is no less than about 96%. In certain embodiments, the enantiomeric excess is no less than about 97%. In certain embodiments, the enantiomeric excess is no less than about 98%. In certain embodiments, the enantiomeric excess is no less than about 99%. In certain embodiments, the enantiomeric excess is no less than about 99.5%. In certain embodiments, the enantiomeric excess is no less than about 99.9%. In certain embodiments, the enantiomeric excess is no less than about 99.95%. In certain embodiments, the enantiomeric excess is no less than about 99.99%. In certain embodiments, the enantiomeric excess is about 100%.

In certain embodiments, the optically active (−)-O-exo/C-exo-tricyclo[5.2.1.0^{2,6}]-dec-9-yl-xanthic acid 1A, or a pharmaceutically acceptable salt, solvate, or prodrug thereof, contains no less than about 60%, no less than about 70%, no less than about 80%, no less than about 90%, no less than about 95%, no less than about 96%, no less than about 97%, no less than about 98%, no less than about 99%, no less than about 99.5%, or no less than about 99.8%, no less than about 99.9%, or about 100% by weight of the (−)-enantiomer.

In certain embodiments, the content of the (−)-enantiomer in the optically active (−)-O-exo/C-exo-tricyclo[5.2.1.0^{2,6}]-dec-9-yl-xanthic acid 1A, or a pharmaceutically acceptable salt, solvate, or prodrug thereof, is no less than about 60% by weight. In certain embodiments, the content of the (−)-enantiomer is no less than about 70% by weight. In certain embodiments, the content of the (−)-enantiomer is no less than about 80% by weight. In certain embodiments, the content of the (−)-enantiomer is no less than about 90% by weight. In certain embodiments, the content of the (−)-enantiomer is no less than about 95% by weight. In certain embodiments, the content of the (−)-enantiomer is no less than about 96% by weight. In certain embodiments, the content of the (−)-enantiomer is no less than about 97% by weight. In certain embodiments, the content of the (−)-enantiomer is no less than about 98% by weight. In certain embodiments, the content of the (−)-enantiomer is no less than about 99% by weight. In certain embodiments, the content of the (−)-enantiomer is no less than about 99.5% by weight. In certain embodiments, the content of the (−)-enantiomer is no less than about 99.8% by weight. In certain embodiments, the content of the (−)-enantiomer is no less than about 99.9% by weight. In certain embodiments, the content of the (−)-enantiomer is about 100% by weight.

In yet another embodiment, provided herein is a pharmaceutically acceptable salt of the optically active (−)-O-exo/C-exo-tricyclo[5.2.1.0^{2,6}]-dec-9-yl-xanthic acid 1A. Suitable bases for use in the preparation of the pharmaceutically acceptable salt, include, but are not limited to, inorganic bases, including, but not limited to, lithium hydroxide, sodium hydroxide, potassium hydroxide, calcium hydroxide, magnesium hydroxide, zinc hydroxide, and ammonium hydroxide; and organic bases, such as primary, secondary, tertiary, and quaternary, aliphatic and aromatic amines, including, but not limited to, L-arginine, benethamine, benzathine, N,N-dibenzylethylenediamine, N-benzylphenethylamine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, chloroprocaine, procaine, N-methyl-D-glucamine, 2-amino-2-(hydroxymethyl)-1,3-propanediol, 1-para-chlorobenzyl-2-pyrrolidin-1′-ylmethyl-benzimidazole, tris(hydroxymethyl)aminomethane, and tromethamine. For reviews on additional pharmaceutically acceptable bases, see, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002.

In certain embodiments, the pharmaceutically acceptable salt of optically active (−)-O-exo/C-exo-tricyclo[5.2.1.0^{2,6}]-dec-9-yl-xanthic acid 1A is an inorganic salt, including, but not limited to, alkali metal, such as lithium, sodium, and potassium; alkali earth metal, such as calcium and magnesium; zinc; and ammonium salts. In certain embodiments, the pharmaceutically acceptable salt is an alkali metal salt. In certain embodiments, the pharmaceutically acceptable salt is an alkali earth metal salt. In certain embodiments, the pharmaceutically acceptable salt is lithium salt. In certain embodiments, the pharmaceutically acceptable salt is magnesium salt. In certain embodiments, the pharmaceutically acceptable salt is calcium salt. In certain embodiments, the pharmaceutically acceptable salt is sodium salt. In certain embodiments, the pharmaceutically acceptable salt is potassium salt. In certain embodiments, the pharmaceutically acceptable salt is zinc salt. In certain embodiments, the pharmaceutically acceptable salt is ammonium salt.

In another embodiment, the pharmaceutically acceptable salt of optically active (−)-O-exo/C-exo-tricyclo[5.2.1.0^{2,6}]-dec-9-yl-xanthic acid 1A is an organic salt. Suitable organic bases for use in the preparation of the pharmaceutically acceptable salt are those as described herein.

In still another embodiment, provided herein is a prodrug of optically active (−)-O-exo/C-exo-tricyclo[5.2.1.0^{2,6}]-dec-9-yl-xanthic acid 1A, including, but not limited to, those disclosed in WO 2005/032492, which is incorporated herein by reference in its entirety.

A prodrug of a compound is a functional derivative of the parent compound which is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

In one embodiment, the prodrug of optically active (−)-O-exo/C-exo-tricyclo[5.2.1.0^{2,6}]-dec-9-yl-xanthic acid 1A is a compound of Formula (I):

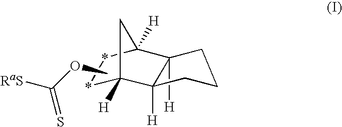

or a pharmaceutically acceptable salt or solvate thereof, wherein R^{a} is alkyl substituted with one or more heteroatoms selected from S, O, N, and P.

In another embodiment, the prodrug is a compound of Formula (II):

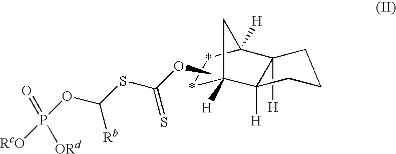

or a pharmaceutically acceptable salt or solvate thereof, wherein R^{b}, R^{c}, and R^{d} are each independently H or alkyl.

The synthesis of the prodrug of Formula (II) is exemplified in Scheme 2 (Krise et al., J. Pharm. Sci. 1999, 88, 922-927; and Krise et al., J. Pharm. Sci. 1999, 88, 928-932). Substitution reaction of compound 2 with potassium salt of optically active (−)-O-exo/C-exo-tricyclo[5.2.1.0^{2,6}]-dec-9-yl-xanthic acid 1A generates the corresponding optically active prodrug of Formula (II). In certain embodiments, R^{b} is H. In certain embodiments, R^{c} and R^{d} are each independently methyl, ethyl, n-propyl, isopropyl, n-butyl, isobutyl, or t-butyl. In certain embodiments, R^{c} and R^{d} are each independently methyl, n-propyl, or t-butyl. In certain embodiments, R^{c} and R^{d} are both methyl. In certain embodiments, R^{c} and R^{d} are both n-propyl. In certain embodiments, R^{c} and R^{d} are both t-butyl. In certain embodiments, R^{b} is H, and R^{c} and R^{d} are each independently methyl, ethyl, n-propyl, isopropyl, n-butyl, isobutyl, or t-butyl.

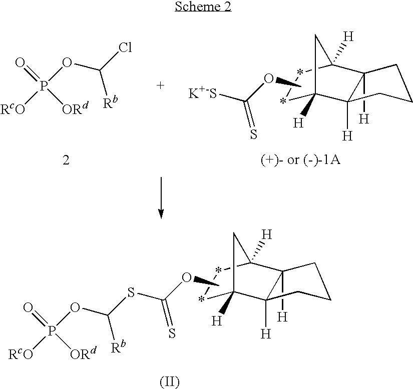

In yet another embodiment, the prodrug is a compound of Formula (III):

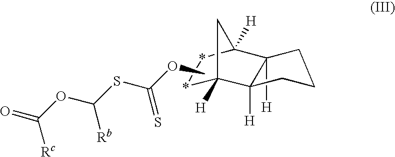

or a pharmaceutically acceptable salt or solvate thereof, wherein R^{b} and R^{c} are each independently H or alkyl. In certain embodiments, R^{b} is H. In certain embodiments, R^{c} is methyl, ethyl, n-propyl, isopropyl, n-butyl, isobutyl, or t-butyl. R^{c} is methyl, n-propyl, or t-butyl. In certain embodiments, R^{b} is H, and R^{c} is methyl, n-propyl, or t-butyl.

The synthesis of the prodrug of Formula (III) is exemplified in Scheme 3. Chloromethyl acetate 3, which is synthesized according to Bodor et al. (J. Org. Chem. 1983, 48, 5280-5284), reacts with potassium salt of optically active (−)-O-exo/C-exo-tricyclo[5.2.1.0^{2,6}]-dec-9-yl-xanthic acid 1A to generate the corresponding optically active prodrug 4 of Formula (III), wherein R^{b} is H.

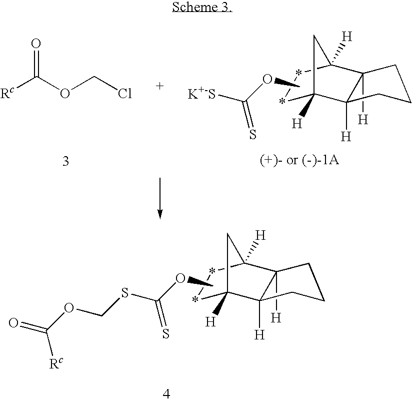

### Preparation of Optically Active (−)-O-exo/C-exo-Tricyclo[5.2.1.0^{2,6}]-dec-9-yl-xanthic Acid

### 1. Asymmetric Hydrosilylation:

Provided herein is an asymmetric hydrosilylation method for the synthesis of optically active (−)-O-exo/C-exo-tricyclo[5.2.1.0^{2,6}]-dec-9-yl-xanthic acid 1A, or a pharmaceutically acceptable salt, solvate, or prodrug thereof (Scheme 4). As used herein, the star symbol “*” in a structure indicates the possible attachment positions for a group so that, when the group is attached to the desirable position, the resulting optically active compound will have an optical activity in the same direction as desired, either (+) or (−).

The method is illustrated herein with the synthesis of optically active (−)-O-exo/C-exo-tricyclo[5.2.1.0^{2,6}]-dec-9-yl-xanthic acid 1A, or a pharmaceutically acceptable salt or solvate thereof. If desired, the method is equally applicable to the synthesis of optically active (+)-O-exo/C-exo-tricyclo[5.2.1.0^{2,6}]-dec-9-yl-xanthic acid 1A, or a pharmaceutically acceptable salt or solvate thereof.

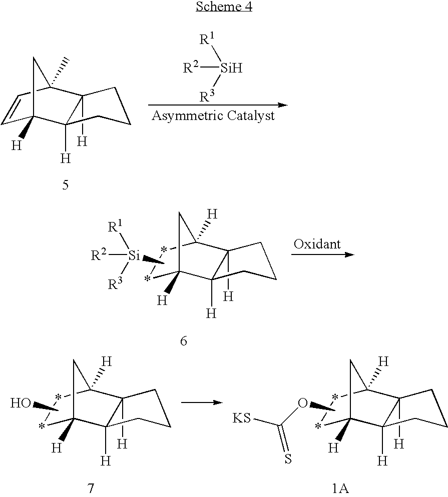

In one embodiment, the method comprises reacting achiral C-exo alkene 5 with a silane in the presence of a transition metal catalyst complexed with a chiral monodentate phosphine to produce optically active organosilane 6.

In another embodiment, the method further comprises oxidizing the optically active organosilane 6 with an oxidant to produce optically active alkanol 7 with the retention of stereochemistry.

In yet another embodiment, the method further comprises converting the optically active alkanol 7 to optically active (−)-O-exo/C-exo-tricyclo[5.2.1.0^{2,6}]-dec-9-yl-xanthic acid 1A, or a pharmaceutically acceptable salt or solvate thereof.

In still another embodiment, the method comprises the steps of: a) reacting achiral C-exo alkene 5 with a silane in the presence of a transition metal catalyst complexed with a chiral monodentate phosphine to produce optically active organosilane 6; b) oxidizing the optically active organosilane 6 with an oxidant to produce optically active alkanol 7 with the retention of stereochemistry; and c) converting the optically active alkanol 7 to optically active (−)-O-exo/C-exo-tricyclo[5.2.1.0^{2,6}]-dec-9-yl-xanthic acid 1A, or a pharmaceutically acceptable salt or solvate thereof.

Suitable silanes for use in the asymmetric hydrosilylation reaction include, but are not limited to, compounds of Formula (IV):

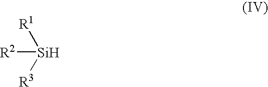

wherein R¹, R², and R³ are each independently H; halogen; C₁₋₆ alkyl, optionally substituted with one or more substituents Q, in one embodiment, one, two, or three substituents Q; or —OR⁴, where R⁴ is C₁₋₆ alkyl, C₃₋₇ cycloalkyl, or C₆₋₁₀ aryl, each optionally substituted with one or more substituents Q, in one embodiment, one, two, or three substituents Q.

Examples of suitable silanes include, but are not limited to, trichlorosilane, methyldichlorosilane, dimethylchlorosilane, methoxydichlorosilane, triethylsilane, pentamethyldisiloxane (HSiMe₂OTMS), and 1,1-dimethyl-3,3-diphenyl-3-tert-butyldisiloxane (HSiMe₂OTBDPS). In certain embodiments, the silane is trichlorosilane. In certain embodiments, the silane is methyldichlorosilane. In certain embodiments, the silane is dimethylchlorosilane. In certain embodiments, the silane is methoxydichlorosilane. In certain embodiments, the silane is triethylsilane. In certain embodiments, the silane is pentamethyldisiloxane (HSiMe₂OTMS). In certain embodiments, the silane is 1,1-dimethyl-3,3-diphenyl-3-tert-butyldisiloxane (HSiMe₂OTBDPS).

Suitable transition metals for use as a catalyst in the asymmetric hydrosilylation reaction include, but are not limited to, platinum, iridium, palladium, rhodium, and ruthenium. The transition metal catalyst can be heterogeneous or homogeneous. In certain embodiments, the transition metal catalyst is platinum. In certain embodiments, the transition metal catalyst is iridium. In certain embodiments, the transition metal catalyst is palladium. In certain embodiments, the transition metal catalyst is rhodium. In certain embodiments, the transition metal catalyst is ruthenium.

Suitable chiral monodentate phosphine ligands include, but are not limited to, compounds of Formula (V):

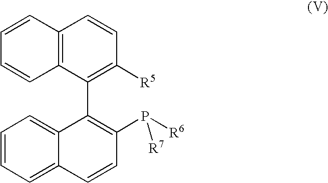

wherein

R⁵ is H; C₁₋₆ alkyl, optionally substituted with one or more substituents Q, in one embodiment, one, two, or three substituents Q; or —OR⁸, where R⁸ is C₁₋₆ alkyl, C₃₋₇ cycloalkyl, or C₆₋₁₀ aryl, each optionally substituted with one or more substituents Q, in one embodiment, one, two, or three substituents Q; and

R⁶ and R⁷ each are independently C₆₋₁₀ aryl, optionally substituted with one or more substituents Q, in one embodiment, one, two, three, four, or five substituents Q.

In certain embodiments, R⁵ is alky, including, but not limited to, methyl, ethyl, n-propyl, isopropyl, n-butyl, isobutyl, t-butyl, pentyl, and hexyl. In certain embodiments, R⁵ is methyl, ethyl, n-propyl, isopropyl, n-butyl, isobutyl, or t-butyl. In certain embodiments, R⁵ is —OR⁸, where R⁸ is C₁₋₆ alkyl, C₃₋₇ cycloalkyl, or C₆₋₁₀ aryl. In certain embodiments, R⁸ is methyl, ethyl, n-propyl, isopropyl, n-butyl, isobutyl, t-butyl, pentyl, or hexyl. In certain embodiments, R⁵ is methyl, ethyl, n-propyl, isopropyl, n-butyl, isobutyl, or t-butyl. In certain embodiments, R⁶ and R⁷ each are independently phenyl; or mono-, di-, tri-, tetra-, or penta-halogenated phenyl. In certain embodiments, R⁶ and R⁷ are phenyl. In certain embodiments, R⁵ is —OR⁸, where R⁸ is methyl. In certain embodiments R⁵ is —OR⁸, where R⁸ is methyl; and R⁶ and R⁷ are phenyl.

The chirality of O-exo/C-exo-tricyclo[5.2.1.0^{2,6}]-dec-9-yl-xanthic acid 1A, or a pharmaceutically acceptable salt, solvate, or prodrug thereof, is determined primarily by the chirality of the chiral monodentate phosphine used. For example, palladium complexed with (R)-(+)-monodentate phosphine ligand of Formula II, wherein R⁵ is methoxy, and R⁶ and R⁷ are phenyl, leads to the formation of optically active (−)-O-exo/C-exo-tricyclo[5.2.1.0^{2,6}]-decan-9-ol (7), which leads to the formation of optically active (−)-O-exo/C-exo-tricyclo[5.2.1.0^{2,6}]-dec-9-yl-xanthic acid 1A, or a pharmaceutically acceptable salt, solvate, or prodrug thereof, with the retention of stereochemistry.

Suitable oxidants for use in the oxidation of the optically active organosilane 6 include, but are not limited to, hydrogen peroxide; and peracids, such as peracetic acid (AcOOH) and m-chloroperbenzoic acid. In certain embodiments, the oxidant is hydrogen peroxide. In certain embodiments, the oxidant is a peracid. In certain embodiments, the oxidant is peracetic acid or m-chloroperbenzoic acid.

The optically active (−)-O-exo/C-exo-tricyclo[5.2.1.0^{2,6}]-decan-9-ol (7) is readily converted into optically active (−)-O-exo/C-exo-tricyclo[5.2.1.0^{2,6}]-dec-9-yl-xanthic acid 1A, or a pharmaceutically acceptable salt, solvate, or prodrug thereof, using the methods known to those skilled in the art, see “Xanthates and Related Compounds,” Rao Ed., Marcel Dekker, Inc., New York, 1971, pgs. 7-31; U.S. Pat. No. 4,602,037; and Gonzalez-Roura et al., Lipids 2002, 37, 401-406.

The starting material, achiral C-exo alkene 5 is prepared from dicyclopentadiene 8 as shown in Scheme 5. Dicyclopentadiene 8 is first treated with hydrobromic acid to generate C-exo-bromoalkene 9 through Wagner-Meerwein rearrangement (Brunson et al., J. Am. Chem. Soc. 1945, 67, 1178-1180). C-exo-Bromoalkene 9 is hydrogenated to provide C-exo bromoalkane 10, which is then dehydrobromated to form achiral C-exo alkene 5 (Youngblood et al., J. Org. Chem. 1956, 21, 1436-1438; Osawa et al., J. Org. Chem. 1982, 47, 1923-1932).

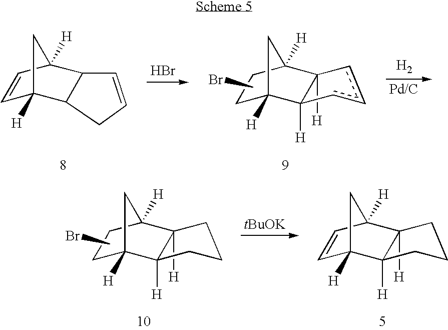

### 2. Enzymatic Resolution: Method I

Provided herein also is a method for the synthesis of optically active (−)-O-exo/C-exo-tricyclo[5.2.1.0^{2,6}]-dec-9-yl-xanthic acid 1A, or a pharmaceutically acceptable salt, solvate, or prodrug thereof, through enzymatic resolution of unsaturated ester 11:

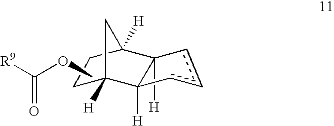

wherein R^{g}C(O)— is C₁₋₂₄ acyl.

The structure of Formula II represents two pairs of enantiomers, (R)-11A and (S)-11A, and (R)-11B and (S)-11B. In certain embodiments, ester 11 used in the enzymatic resolution is a mixture of all four stereoisomers, (R)-11A, (S)-11A, (R)-11B, and (S)-11B. In certain embodiments, ester 11 used in the enzymatic resolution is a mixture of (R)-11A and (S)-11A, such as a racemic mixture thereof. In certain embodiments, ester 11 used in the enzymatic resolution is a mixture of (R)-11B and (S)-11B, such as a racemic mixture thereof.

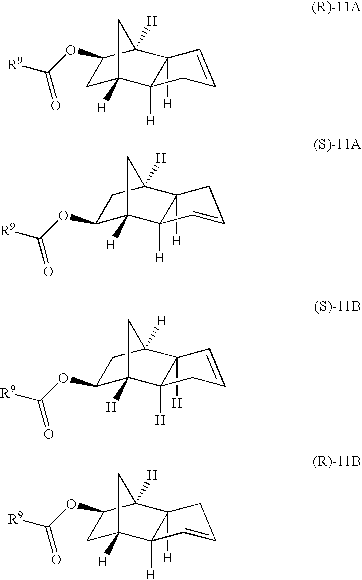

In certain embodiments, the acyl is acetyl, propionyl, butanoyl, isobutanoyl, pentanoyl, hexanoyl, heptanoyl, octanoyl, nonanoyl, decanoyl, dodecanoyl, tetradecanoyl, hexadecanoyl, octadecanoyl, eicosanoyl, docosanoyl, myristoleoyl, palmitoleoyl, oleoyl, linoleoyl, or arachidonoyl. In certain embodiments, the acyl is acetyl. In certain embodiments, the acyl is propionyl. In certain embodiments, the acyl is butanoyl. In certain embodiments, the acyl is isobutanoyl. In certain embodiments, the acyl is pentanoyl. In certain embodiments, the acyl is hexanoyl. In certain embodiments, the acyl is heptanoyl. In certain embodiments, the acyl is octanoyl. In certain embodiments, the acyl is nonanoyl. In certain embodiments, the acyl is decanoyl. In certain embodiments, the acyl is dodecanoyl. In certain embodiments, the acyl is tetradecanoyl. In certain embodiments, the acyl is hexadecanoyl. In certain embodiments, the acyl is octadecanoyl. In certain embodiments, the acyl is eicosanoyl. In certain embodiments, the acyl is docosanoyl. In certain embodiments, the acyl is myristoleoyl. In certain embodiments, the acyl is palmitoleoyl. In certain embodiments, the acyl is oleoyl. In certain embodiments, the acyl is linoleoyl. In certain embodiments, the acyl is arachidonoyl. In certain embodiments, the acyl is a natural fatty acyl, including, but not limited to, butanoyl, hexanoyl, octanoyl, decanoyl, dodecanoyl, tetradecanoyl, hexadecanoyl, octadecanoyl, eicosanoyl, docosanoyl, myristoleoyl, palmitoleoyl, oleoyl, linoleoyl, and arachidonoyl.

In one embodiment, the method comprises the step of selectively hydrolyzing unsaturated ester 11 with a hydrolytic enzyme to produce optically active (+)- or (−)-alkenol 12, depending on the specificity of the enzyme, and leaving the other enantiomer as optically active unreacted ester 11 with an opposite optical activity (Scheme 6). If the desired enantiomer is in ester form, the method may also comprise the step of separating the optically active alkenol 12 from the optically active unreacted ester 11 after enzymatic hydrolysis, using conventional methods, such as chromatography. When the desired enantiomer is the optically active unreacted ester 11, the method further comprises the step of converting the optically active ester 11 to the corresponding optically active alcohol 12, which can be accomplished using conventional methods known to those skilled in the art, for example, treating the optically active ester 11 with a base, such as lithium hydroxide, sodium hydroxide, or potassium hydroxide.

As used herein, the term “hydrolytic enzyme” refers to a hydrolytic enzyme or a microorganism that contains the hydrolytic enzyme. The hydrolytic enzyme may be obtained from any sources, including, but not limited to, animals, plants, and microorganisms. The enzyme may be employed in any conventional form, such as in a purified form, a crude form, a microbial fermentation broth, a fermentation broth, or a filtrate of fermentation broth. In addition, the enzyme or microorganism may be immobilized.

Suitable hydrolytic enzymes for use in the enzymatic resolution include, but are not limited to, lipases, esterases, peptidases, amidases, and acylases.

Suitable lipases include, but are not limited to Amano PS-30 (Pseudomonas cepacla), Amano GC-20 (Geotrichum candidum), Amano APF (Aspergillus niger), Amano AK (Pseudomonas sp.), Pseudomonas fluorescens lipase, Amano Lipase P30 (Pseudomonas sp.), Amano P (Pseudomonas fluorescens), Amano AY-30 (Candida cylindracea), Amano N (Rhizopus niveus), Amano R (Penicillium sp.), Amano FAP (Rhizopus oryzae), Amano AP-12 (Aspergillus niger), Amano MAP (Mucor melhei), Amano GC-4 (Geotrichum candidum), Sigma L-0382 and L-3126 (porcine pancrease), Lipase OF, Lipase R (Rhizopus sp.), Sigma L-3001 (Wheat germ), Sigma L-1754 (Candida cytindracea), Sigma L-0763 (Chromobacterium viscosum), Amano K-30 (Aspergillus niger), and Candida antactica lipase A. or Pseudomonas fluorescens lipase. Suitable peptidases include, but are not limited to, Rhizopus oryzae peptidase.

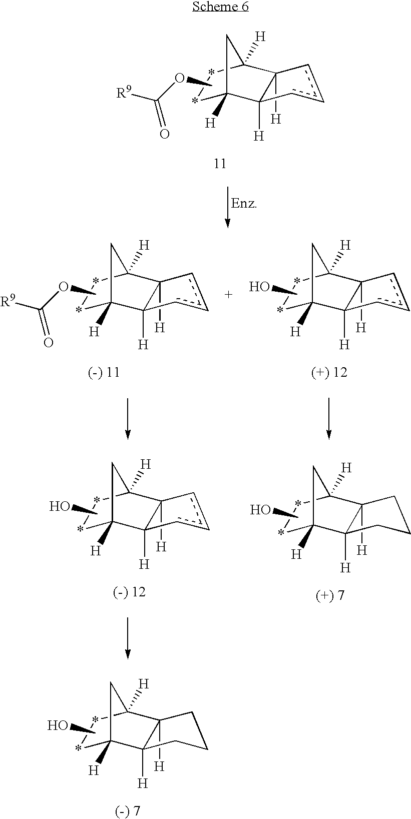

In certain embodiments, the enzymatic resolution is performed in a buffer solution, including inorganic acid salt buffers, such as potassium dihydrogen phosphate or sodium dihydrogen phosphate; and organic acid salt buffers, such as sodium citrate. The concentration of the buffer may vary from about 0.005 to about 2 M or about 0.005 to about 0.5 M, and depend on the specific enzymes or microorganism used.

Depending on the solubility of ester 11, a surfactant may be added to the reaction mixture to solubilize the substrate. Suitable surfactants include, but are not limited to, nonionic surfactants, such as alkylaryl polyether alcohols, octylphenoxy polyethoxyethanol, and Triton X-100.

An organic solvent may also added as co-solvent to facilitate the enzymatic resolution. Suitable solvents include, but are not limited to, acetonitrile, t-butyl methyl ether, THF, DMSO, DMF, and alcohols.

In another embodiment, the method is for preparing (−)-O-exo/C-exo-tricyclo[5.2.1.0^{2,6}]-dec-9-yl-xanthic acid 1A, or a pharmaceutically acceptable salt, solvate, or prodrug thereof, as shown in Scheme 6, which comprises the steps of: a) selectively hydrolyzing ester 11 with a hydrolytic enzyme to produce optically active (−)-ester 11 and optically active (+)-alkenol 12; b) hydrolyzing the optically active (−)-ester 11 to produce optically active (−)-alkenol 12; c) reducing the optically active (−)-alkenol 12 to produce optically active (−)-alkanol 7 with the retention of stereochemistry; and d) converting the optically active (−)-alkanol 7 to optically active (−)-O-exo/C-exo-tricyclo[5.2.1.0^{2,6}]-dec-9-yl-xanthic acid 1A, or a pharmaceutically acceptable salt, solvate, or prodrug thereof.

In the method provided herein, the hydrolyzing reaction (step b) and reduction reaction (step c) are not limited to any particular order. If desired, the reduction reaction can be carried out prior to the hydrolyzing reaction.

If desired, (+)-O-exo/C-exo-tricyclo[5.2.1.0^{2,6}]-dec-9-yl-xanthic acid 1A, or a pharmaceutically acceptable salt, solvate, or prodrug thereof, can also be prepared similarly. The method for the synthesis of the (+) enantiomer comprises the steps of: a) selectively hydrolyzing ester 11 with a hydrolytic enzyme to produce optically active (−)-ester 11 and optically active (+)-alkenol 12; b) reducing the optically active (+)-alkenol 12 to produce optically active (+)-alkanol 7 with the retention of stereochemistry; and c) converting the optically active (+)-alkanol 7 to optically active (+)-O-exo/C-exo-tricyclo[5.2.1.0^{2,6}]-dec-9-yl-xanthic acid 1A, or a pharmaceutically acceptable salt, solvate, or prodrug thereof.

In certain embodiments, the hydrolytic enzyme is a lipase, esterase, peptidase, amidase, or acylase. In certain embodiments, the hydrolytic enzyme is a lipase. In certain embodiments, the hydrolytic enzyme is a peptidase. In certain embodiments, the hydrolytic enzyme is an esterase. In certain embodiments, the hydrolytic enzyme is an amidase. In certain embodiments, the hydrolytic enzyme is an acylase. In certain embodiments, the hydrolytic enzyme is Rhizopus oryzae peptidase, Candida antactica lipase A, or Pseudomonas fluorescens lipase, each optionally immobilized. In certain embodiments, the hydrolytic enzyme is Rhizopus oryzae peptidase, Candida antactica lipase A, or immobilized Candida antactica lipase A. In certain embodiments, the hydrolytic enzyme is Rhizopus oryzae peptidase. In certain embodiments, the hydrolytic enzyme is Candida antactica lipase A. In certain embodiments, the hydrolytic enzyme is immobilized Candida antactica lipase A.

In certain embodiments, the enzyme used in the enzymatic resolution is in a catalytic amount, that is, the amount of the enzyme in the enzymatic resolution reaction is no greater than about 50%, no greater than about 25%, no greater than about 20%, no greater than about 15%, or no greater than about 10% by weight of ester 11. In certain embodiments, the enzyme used in the enzymatic resolution is no greater than bout 50% by weight of ester 11. In certain embodiments, the enzyme used in the enzymatic resolution is no greater than bout 25% by weight of ester 11. In certain embodiments, the enzyme used in the enzymatic resolution is no greater than bout 20% by weight of ester 11. In certain embodiments, the enzyme used in the enzymatic resolution is no greater than bout 15% by weight of ester 11. In certain embodiments, the enzyme used in the enzymatic resolution is no greater than bout 10% by weight of ester 11.

In certain embodiments, the enzymatic resolution is carried out at a temperature from about 5 to about 100° C., from about 10 to about 75° C., from about 15 to 60° C., from about 20 to about 50° C., from about 25 to about 40° C., or from about 30 to about 40° C. In certain embodiments, the temperature is from about 5 to about 100° C. In certain embodiments, the temperature is from about 10 to about 75° C. In certain embodiments, the temperature is from about 15 to about 60° C. In certain embodiments, the temperature is from about 20 to about 50° C. In certain embodiments, the temperature is from about 25 to about 40° C. In certain embodiments, the temperature is from about 30 to about 40° C.

In certain embodiments, the enzymatic resolution is carried out for duration of time of no greater than about 48 hrs, no greater than about 36 hrs, or no greater than about 24 hrs.

The starting material, O-exo/C-exo-ester 11, is prepared from dicyclopentadiene 8 as shown in Scheme 7. Dicyclopentadiene 8 is first treated with sulfuric acid to form O-exo/C-exo-alkenol 12 (Brunson and Riener, J. Am. Chem. Soc. 1945, 67, 723-728), followed by acylation to yield the starting material O-exo/C-exo-ester 11.

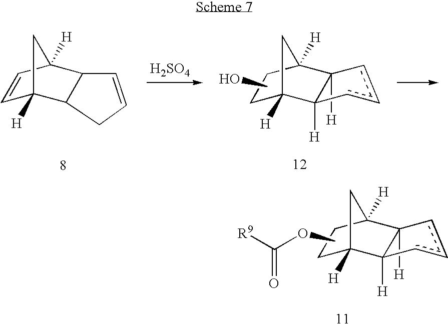

### 3. Enzymatic Resolution: Method II

In yet another embodiment, optically active (−) O-exo/C-exo-tricyclo[5.2.1.0^{2,6}]-dec-9-yl-xanthic acid 1A, or a pharmaceutically acceptable salt, solvate, or prodrug thereof, is prepared through enzymatic resolution of ester 13:

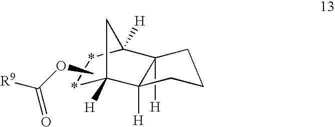

wherein R⁹C(O)— is as described herein. The structure of Formula 13 represents two enantiomers, (R)-13 and (S)-13.

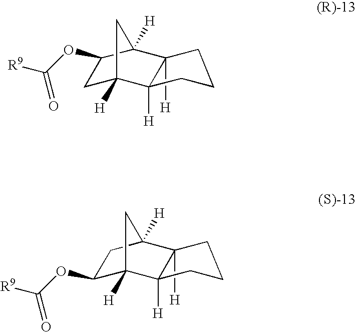

In one embodiment, the method comprises the step of selectively hydrolyzing ester 13 with a hydrolytic enzyme to produce optically active (+)- or (−)-alkanol 7, depending on the specificity of the enzyme, and leaving the other enantiomer as optically active unreacted ester 13 with an opposite optical activity. The method may also comprise the step of separating the optically active alkanol 7 from the optically active unreacted ester 13 after enzymatic hydrolysis, using conventional methods, such as chromatography. When the desired enantiomer is the optically active unreacted ester 13, the method further comprises the step of converting the optically active ester 13 to the corresponding optically active alcohol 7, which can be accomplished using conventional methods known to those skilled in the art, for example, treating the optically active ester 13 with a base, such as lithium hydroxide, sodium hydroxide, and potassium hydroxide. Suitable reaction conditions and parameters, such as the hydrolytic enzyme, solvent, and buffer are those as described herein.

In another embodiment, the method for preparing (−)-O-exo/C-exo-tricyclo[5.2.1.0^{2,6}]-dec-9-yl-xanthic acid 1A, or a pharmaceutically acceptable salt, solvate, or prodrug thereof, comprises the steps of: a) selectively hydrolyzing ester 13 with a hydrolytic enzyme to produce optically active (−)-ester 13 and optically active (+)-alkanol 7; b) hydrolyzing the optically active (−)-ester 13 to produce optically active (−)-alkanol 7; and c) converting the optically active (−)-alkanol 7 to optically active (−)-O-exo/C-exo-tricyclo[5.2.1.0^{2,6}]-dec-9-yl-xanthic acid 1A, or a pharmaceutically acceptable salt, solvate, or prodrug thereof.

If desired, (+)-O-exo/C-exo-tricyclo[5.2.1.0^{2,6}]-dec-9-yl-xanthic acid 1A, or a pharmaceutically acceptable salt, solvate, or prodrug thereof, can also be prepared similarly. The method for the synthesis of the (+) enantiomer comprises the steps of: a) selectively hydrolyzing ester 13 with a hydrolytic enzyme to produce optically active (−)-ester 13 and optically active (+)-alkanol 7; and b) converting the optically active (+)-alkanol 7 to optically active (+)-O-exo/C-exo-tricyclo[5.2.1.0^{2,6}]-dec-9-yl-xanthic acid 1A, or a pharmaceutically acceptable salt, solvate, or prodrug thereof.

In certain embodiments, the hydrolytic enzyme is Rhizopus oryzae peptidase, Candida antactica lipase A, or Pseudomonas fluorescens lipase, each optionally immobilized. In certain embodiments, the hydrolytic enzyme is Rhizopus oryzae peptidase, Candida antactica lipase A, or Pseudomonas fluorescens lipase. In certain embodiments, the hydrolytic enzyme is Rhizopus oryzae peptidase. In certain embodiments, the hydrolytic enzyme is Candida antactica lipase A. In certain embodiments, the hydrolytic enzyme is Pseudomonas fluorescens lipase.

The starting material, ester 13 is prepared from O-exo/C-exo-alkenol 12 as shown in Scheme 8. O-exo/C-exo-Alkenol 12 is hydrogenated to O-exo/C-exo-alkanol 7, followed by acylation to yield the starting material O-exo/C-exo-ester 13.

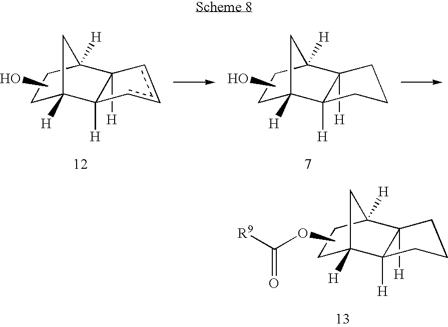

The optically active O-exo/C-exo-tricyclo[5.2.1.0^{2,6}]-dec-9-yl-xanthic acid 1A, or a pharmaceutically acceptable salt, solvate, or prodrug thereof, may also be prepared using other conventional methods and techniques known to those skilled in the art. For example, racemic O-exo/C-exo-tricyclo[5.2.1.0^{2,6}]-dec-9-yl-xanthic acid 1A may be resolved by reacting with an optically active base to form diastereomers, followed by chromatography or fractional crystallization, and regeneration of the free acid or conversion into a pharmaceutically acceptable salt, solvate, or prodrug thereof.

Alternatively, racemic O-exo/C-exo-tricyclo[5.2.1.0^{2,6}]-dec-9-yl-xanthic acid 1A, or a pharmaceutically acceptable salt, solvate, or prodrug thereof, may be resolved chromatographically using a chiral column or TLC. Various chiral columns and eluents for use in the separation of the enantiomers are available and suitable conditions for the separation can be empirically determined by methods known to one of skill in the art. Exemplary chiral columns available for use in the separation of the enantiomers provided herein include, but are not limited to, CHIRALCEL® OB, CHIRALCEL® OB-H, CHIRALCEL® OD, CHIRALCEL® OD-H, CHIRALCEL® OF, CHIRALCEL® OG, CHIRALCEL® OJ, and CHIRALCEL® OK.

Additional methods and technologies can be found in, e.g., Enantiomers, Racemates and Resolutions, Jacques et al., Wiley-Interscience, New York, 1981; Wilen, Collet, and Jacques, Tetrahedron 1977, 2725-2736; Stereochemistry of Carbon Compounds, Eliel, McGraw-Hill, New York, 1962; Wilen in Tables of Resolving Agents and Optical Resolutions, Eliel, Ed., Univ. of Notre Dame Press, Notre Dame, Indianapolis, 1972, pgs. 268-298; Stereochemistry of Organic Compounds, Eliel, Wilen, and Manda, John Wiley & Sons, Inc., 1994; and Stereoselective Synthesis A Practical Approach, Nógrádi, VCH Publishers, Inc., New York, 1995.

The identity and optical purity of an optically active stereoisomer of O-exo/C-exo-tricyclo[5.2.1.0^{2,6}]-dec-9-yl-xanthic acid 1A, or a pharmaceutically acceptable salt, solvate, or prodrug thereof; and chiral intermediates, such as compounds 6, 7, and 11 to 13, can be determined by polarimetry, NMR, or other analytical methods known in the art.

### Pharmaceutical Compositions

Provided herein are pharmaceutical compositions, which comprise the optically active (−)-O-exo/C-exo-tricyclo[5.2.1.0^{2,6}]-dec-9-yl-xanthic acid 1A, or a pharmaceutically acceptable salt, solvate, or prodrug thereof, as an active ingredient, in combination with one or more pharmaceutically acceptable excipients or carriers. In certain embodiments, the pharmaceutical composition comprises at least one release controlling excipient or carrier. In certain embodiments, the pharmaceutical composition comprises at least one nonrelease controlling excipient or carrier. In certain embodiments, the pharmaceutical composition comprises at least one release controlling and at least one nonrelease controlling excipients or carriers.

The pharmaceutical compositions provided herein may be provided in unit-dosage forms or multiple-dosage forms. Unit-dosage forms, as used herein, refer to physically discrete units suitable for administration to human and animal subjects, and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampouls, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The optically active (−)-O-exo/C-exo-tricyclo[5.2.1.0^{2,6}]-dec-9-yl-xanthic acid 1A, and a pharmaceutically acceptable salt, solvate, and prodrug thereof, may be administered alone, or in combination with one or more other active ingredients. The pharmaceutical compositions provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

In one embodiment, the pharmaceutical compositions are provided as a dosage form for oral administration to a subject, which comprise optically active (−)-O-exo/C-exo-tricyclo[5.2.1.0^{2,6}]-dec-9-yl-xanthic acid 1A, or a pharmaceutically acceptable salt, solvate, or prodrug thereof, and one or more pharmaceutically acceptable excipients or carriers. The pharmaceutical compositions may also comprise one or more adjuvants to further enhance their pharmacological properties. Suitable adjuvants include, but are not limited to, ionic detergents, lipids, and steroids. Examples of ionic detergents include C₆₋₁₉ fatty acids and salts thereof, such as decanoic, undecanoic, lauric acid, potassium decanate, potassium undecanate, potassium laurate, sodium decanate, sodium undecanate, and sodium laurate; and C₈₋₁₈ alkylsulfate, including sodium laurylsulfate and potassium larylsulfate. Examples of lipids include phospholipids, such as phosphatidylcholine, phosphatidylserine, and phosphatidylinositol; glycolipids, such as ganglisoide; and sphingolipids, such as sphingomyelin. Examples of steroids include stearylamine, cholesterol; cholestanol, cholanic acid, chondrillasterol, and α,β,γ-sisterol.

In another embodiment, the pharmaceutical compositions are provided as a dosage form for parental administration to a subject, which comprise optically active (−)-O-exo/C-exo-tricyclo[5.2.1.0^{2,6}]-dec-9-yl-xanthic acid 1A, or a pharmaceutically acceptable salt, solvate, or prodrug thereof, and one or more pharmaceutically acceptable excipients or carriers. The pharmaceutical compositions may also comprise one or more adjuvants as described herein to further enhance their pharmacological characteristics.

In yet another embodiment, the pharmaceutical compositions are provided as a dosage form for topical administration to a subject, which comprise an optically active (−)-O-exo/C-exo-tricyclo[5.2.1.0^{2,6}]-dec-9-yl-xanthic acid 1A, or a pharmaceutically acceptable salt, solvate, or prodrug thereof, and one or more pharmaceutically acceptable excipients or carriers. The pharmaceutical compositions may also comprise one or more adjuvants as described herein to further enhance their pharmacological characteristics.

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understand that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and H₂-receptor antagonists.

### B. Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including alpha-cyclodextrin, beta-cyclodextrin, hydroxypropyl-beta-cyclodextrin, sulfobutylether-beta-cyclodextrin, and sulfobutylether 7-beta-cyclodextrin (CAPTISOL®, CyDex, Lenexa, Kans.).

The pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon bases, including lard, benzoinated lard, olive oil, cottonseed oil, white petrolatum, and plastibase; emulsifiable or absorption bases, such as hydrophilic petrolatum, hydroxystearin sulfate, and anhydrous lanolin; water-removable bases, such as hydrophilic ointment; water-soluble ointment bases, including polyethylene glycols of varying molecular weight; emulsion bases, either water-in-oil (W/0) emulsions or oil-in-water (0/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein may be micronized to a size suitable for delivery by inhalation, such as 50 micrometers or less, or 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

### D. Modified Release

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile- or pulsed-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In another embodiment, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The pharmaceutical compositions provided herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), hydroxylethyl cellulose, and other pharmaceutically acceptable excipients.

### 3. Multiparticulate Controlled Release Devices

The pharmaceutical compositions provided herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

Provided herein is a method for treating, preventing, or ameliorating a disease caused by a virus, which comprises administering to a subject a therapeutically effective amount of optically active (−)-O-exo/C-exo-tricyclo[5.2.1.0^{2,6}]-dec-9-yl-xanthic acid 1A, or a pharmaceutically acceptable salt, solvate, or prodrug thereof. Examples of the diseases caused by a virus include, but are not limited to, molluscum contagiosum infection, HTLV infection, HTLV-1 infection, HIV infection (AIDS), human papillomavirus infection, herpesvirus infection, genital herpes infection, viral dysentery, flu, measles, rubella, chickenpox, mumps, polio, rabies, mononucleosis, ebola, respiratory syncytial virus infection, dengue fever, yellow fever, lassa fever, arena virus infection, bunyavirus infection, filovirus infection, flavivirus infection, hantavirus infection, rotavirus infection, viral meningitis, west Nile fever, arbovirus infection, parainfluenza, smallpox, epstein-barr virus infection, dengue hemorrhagic fever, cytomegalovirus infection, infant cytomegalic virus infection, progressive multifocal leukoencephalopathy, viral gastroenteritis, hepatitis, cold sores, ocular herpes, meningitis, encephalitis, shingles, encephalitis, california serogroup virus infection, St. Louis encephalitis, rift valley fever, hand, foot, & mouth disease, hendra virus infection, enterovirus infection, astrovirus infection, adenovirus infection, Japanese encephalitis, lymphocytic choriomeningitis, roseola infantum, sandfly fever, SARS, warts, cat scratch disease, slap-cheek syndrome, orf, pityriasis rosea, lyssavirus infection, H5N1 virus infection (bird flu), and human papaloma virus infection.

Examples of the viruses that are amenable to the method for treatment provided herein include, but are not limited to, adenoviruses, arbovirus, arenavirus, astroviruses, bunyaviruses, coronaviruses, Coxsackievirus, cytomegalovirus, dengue virus, ebolavirus, enteroviruses, Epstein-Barr virus, flavivirus, filoviruses, H5N1 virus, hendravirus, human T-lyphotropic viruses, human immunodeficiency viruses, human papillomaviruses, hantaviruses, hepatitis viruses, hepadnavirus, herpesviruses, herpes simplex viruses-1, herpes simplex virus-2, infant cytomegalic virus, influenza viruses, Japanese encephalitis virus, JC virus, lassa virus, lymphocytic choriomeningitis virus, lyssavirus, molluscum contagiosum virus, mumps virus, orf virus, parainfluenza viruses, paramyxovirus, parapoxvirus, parvovirus, picornavirus, poliovirus, polyomavirus, rabies virus, rift valley fever virus, Roseolovirus, rotaviruses, rubella virus, smallpox viruses, St. Louis encephalitis virus, varicella zoster virus, West Nile virus, and yellow fever virus.

In one embodiment, the virus is sexually transmissible. In another embodiment, the virus is an oncogenic virus. In certain embodiments, the virus is papovavirus or herpes simplex virus. In certain embodiments, the papovavirus is a polyoma or papilloma virus. In certain embodiments, the papovavirus is a polyoma virus. In certain embodiments, the papovavirus is papilloma virus. In certain embodiments, the virus is human papilloma virus. In certain embodiments, the virus is herpes simplex virus.

Provided also herein is a method for treating, preventing, or ameliorating one or more symptoms of a disease caused by an oncogenic virus, which comprises administering to a subject having or being suspected to have such a disease, a therapeutically effective amount of optically active (−)-O-exo/C-exo-tricyclo[5.2.1.0^{2,6}]-dec-9-yl-xanthic acid 1A, or a pharmaceutically acceptable salt, solvate, or prodrug thereof.

In certain embodiments, the oncogenic virus is sexually transmissible. In certain embodiments, the oncogenic virus is papovavirus. In certain embodiments, the oncogenic virus is a polyoma or papilloma virus. In certain embodiments, the oncogenic virus is a polyoma virus. In certain embodiments, the oncogenic virus is papilloma virus. In certain embodiments, the oncogenic virus is human or bovine papilloma virus.

In certain embodiments, the disease caused by an oncogenic virus is a wart, including, but not limited to, a plantar wart and genital wart; cervical dysplasia; recurrent respiratory papillomatosis, including, but not limited to, laryngeal papillomas; or a cancer associated with papillomavirus infection, including anogenital cancers, such as cervical, anal and perianal, vulvar, vaginal, and penile cancers; head and neck cancers, such as oral pharyngeal region and esophagus cancers; and skin cancers, such as basal cell carcinoma and squamous cell carcinoma.

In certain embodiments, administration of a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 10%, 20%, 30%, 40%, 50%, 60%, 70%, 80%, 90%, 95%, 99% or more reduction in the replication of the virus relative to a subject without administration of the compound, as determined at 1 day, 2 days, 3 days, 4 days, 5 days, 10 days, 15 days, or 30 days after the administration by a method known in the art, e.g., determination of viral titer.

In certain embodiments, administration of a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 1, 2, 3, 4, 5, 10, 15, 20, 25, 50, 75, 100-fold or more reduction in the replication of the virus relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 10%, 20%, 30%, 40%, 50%, 60%, 70%, 80%, 90%, 95%, 99% or more reduction in the viral titer relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 1, 2, 3, 4, 5, 10, 15, 20, 25, 50, 75, 100 or more fold reduction in the viral titer relative to a subject without administration of the compound, as determined at 1 day, 2 days, 3 days, 4 days, 5 days, 10 days, 15 days, or 30 days after the administration by a method known in the art.

Further provided herein is a method for inhibiting the replication of a virus, which comprises contacting the virus with an effective amount of optically active (−)-O-exo/C-exo-tricyclo[5.2.1.0^{2,6}]-dec-9-yl-xanthic acid 1A, or a pharmaceutically acceptable salt, solvate, or prodrug thereof.

In one embodiment, the virus is sexually transmissible. In another embodiment, the virus is an oncogenic virus. In certain embodiments, the virus is papovavirus or herpes simplex virus. In certain embodiments, the papovavirus is a polyoma or papilloma virus. In certain embodiments, the papovavirus is a polyoma virus. In certain embodiments, the papovavirus is papilloma virus. In certain embodiments, the virus is human papilloma virus. In certain embodiments, the virus is herpes simplex virus.

In certain embodiments, the contacting of the virus with a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 10%, 20%, 30%, 40%, 50%, 60%, 70%, 80%, 90%, 95%, 99% or more reduction in the virus titer relative to the virus without such contact, as determined at 1 day, 2 days, 3 days, 4 days, 5 days, 10 days, 15 days, or 30 days after the initial contact, by a method known in the art.

In certain embodiments, the contacting of the virus with a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 1, 2, 3, 4, 5, 10, 15, 20, 25, 50, 75, 100-fold or more reduction in the virus titer relative to the virus without such contact, as determined at 1 day, 2 days, 3 days, 4 days, 5 days, 10 days, 15 days, or 30 days after the initial contact, by a method known in the art.

In certain embodiments, the contacting of the virus with a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 10%, 20%, 30%, 40%, 50%, 60%, 70%, 80%, 90%, 95%, 99% or more reduction in the viral titer relative to the virus without such contact, as determined at 1 day, 2 days, 3 days, 4 days, 5 days, 10 days, 15 days, or 30 days after the initial contact by a method known in the art.

In certain embodiments, the contacting of the virus with a therapeutically effective amount of the optically active (−)-O-exo/C-exo-tricyclo[5.2.1.0^{2,6}]-dec-9-yl-xanthic acid 1A, or a pharmaceutically acceptable salt, solvate, or prodrug thereof; or a pharmaceutical composition thereof, results in a 1, 2, 3, 4, 5, 10, 15, 20, 25, 50, 75, 100 or more fold reduction in the viral titer relative to the virus without such contact, as determined at 1 day, 2 days, 3 days, 4 days, 5 days, 10 days, 15 days, or 30 days after the initial contact, by a method known in the art.

Provided also herein is a method for treating, preventing, or ameliorating one or more symptoms of a disease in a subject, which comprises administering to the subject having or being suspected to have such a disease, a therapeutically effective amount of optically active (−)-O-exo/C-exo-tricyclo[5.2.1.0^{2,6}]-dec-9-yl-xanthic acid 1A, or a pharmaceutically acceptable salt, solvate, or prodrug thereof.

In one embodiment, the disease is cancer, including, but not limited to, breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular lymphoma, and multiple myeloma.

Provided herein is a method for inhibiting the activity of phospholipase C, which comprises contacting phospholipase C with optically active (−)-O-exo/C-exo-tricyclo[5.2.1.0^{2,6}]-dec-9-yl-xanthic acid 1A, or a pharmaceutically acceptable salt, solvate, or prodrug thereof.

Depending on the disease to be treated and the subject's condition, the optically active (−)-O-exo/C-exo-tricyclo[5.2.1.0^{2,6}]-dec-9-yl-xanthic acid 1A, or a pharmaceutically acceptable salt, solvate, or prodrug thereof, provided herein may be administered by oral, parenteral (e.g., intramuscular, intraperitoneal, intravenous, ICY, intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from 0.1 to 10 milligram, from 0.1 to 5 milligrams, or from 0.1 to 2 milligram active ingredient(s) per dosage unit, and if the condition of the patient requires, the dose can, by way of alternative, be administered as a continuous infusion.

In certain embodiments, an appropriate dosage level is about 0.001 to about 10 mg per kg patient body weight per day (mg/kg per day), about 0.01 to about 10 mg/kg per day, about 0.01 to about 1 mg/kg per day, or about 0.05 to about 1 mg/kg per day, which may be administered in single or multiple doses. A suitable dosage level may be about 0.001 to 25 mg/kg per day, about 0.001 to 10 mg/kg per day, or about 0.001 to 5 mg/kg per day. Within this range the dosage may be 0.001 to 0.005, 0.005 to 0.05, 0.05 to 0.5 or 0.5 to 5.0 mg/kg per day.

In certain embodiments, an appropriate dosage level is about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10 mg/kg per day.

### Kits/Articles of Manufacture

The optically active (−)-O-exo/C-exo-tricyclo[5.2.1.0^{2,6}]-dec-9-yl-xanthic acid 1A, or a pharmaceutically acceptable salt, solvate, or prodrug thereof, can also provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of the optically active (−)-O-exo/C-exo-tricyclo[5.2.1.0^{2,6}]-dec-9-yl-xanthic acid 1A, or a pharmaceutically acceptable salt, solvate, or prodrug thereof.

In certain embodiments, the optically active (−)-O-exo/C-exo-tricyclo[5.2.1.0^{2,6}]-dec-9-yl-xanthic acid 1A, or a pharmaceutically acceptable salt, solvate, or prodrug thereof, is administered in combination with other therapeutic agents as described herein. The other therapeutic agents may or may not be administered to a patient at the same time or by the same route of administration.

In certain embodiments, the kit includes a container comprising a dosage form of the optically active (−)-O-exo/C-exo-tricyclo[5.2.1.0^{2,6}]-dec-9-yl-xanthic acid 1A, or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); L (liters); mL (milliliters); μL, (microliters); psi (pounds per square inch); M (molar); mM (millimolar); μM (micromolar); Hz (Hertz); MHz (megahertz); mol (moles); mmol (millimoles); RT (room temperature); hr (hours); min (minutes); TLC (thin layer chromatography); mp (melting point); RP (reverse phase); Tᵣ (retention time); TFA (trifluoroacetic acid); TEA (triethylamine); THF (tetrahydrofuran); TFAA (trifluoroacetic anhydride); CD₃OD (deuterated methanol); CDCl₃ (deuterated chloroform); DMSO (dimethylsulfoxide); SiO₂ (silica); atm (atmosphere); EtOAc (ethyl acetate); CHCl₃ (chloroform); HCl (hydrochloric acid); Ac (acetyl); DMF (N,N-dimethylformamide); Me (methyl); Cs₂CO₃ (cesium carbonate); EtOH (ethanol); Et (ethyl); tBu (tert-butyl); MeOH (methanol).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies illustrated in Schemes 2 to 6 are intended to exemplify the applicable chemistry through the use of specific examples and are not indicative of the scope of the disclosure.

### Example 1

### Synthesis of optically active (−)-O-exo/C-exo-tricyclo[5.2.1.0^{2,6}]-dec-9-yl-xanthic acid 1A

The synthesis of optically active potassium salt of (−)-O-exo/C-exo-tricyclo[5.2.1.0^{2,6}]-dec-9-yl-xanthic acid 1A is illustrated in Schemes 4 and 5.

Step 1. A mixture of dicyclopentadiene 8 (132.2 g, 1 mol) and 48% hydrobromic acid (227 mL) was stirred at 70° C. for 3 hrs. The precipitates were filtered and washed with hexane. The organic layer was separated from the aqueous layer, and the aqueous layer was further extracted with hexane. The combined organic layers were washed with water, dried over anhydrous MgSO₄, and concentrated. The residual oil was distilled at 105-113° C. under vacuum (12 mmHg) to provide C-exo bromoalkene 9 as colorless oil (199.3 g, 93.5%).

¹H NMR (CDCl₃) δ: 1.30-1.65 (2H, m), 1.75-2.15 (5H, m), 2.25-2.35 (1H, m), 2.40-2.70 (2H, m), 3.95-4.05 (1H, m), 5.60-5.75 (1H, m).

Step 2. To a solution of C-exo bromoalkene 9 in ethyl acetate (200 mL) was added 10% Pd/C (2 g). The mixture was hydrogenated at 50 psi overnight. The catalyst was removed by filtration and the reaction solution was concentrated. The residual oil was distilled at 112-118° C. under vacuum (12 mmHg) to provide C-exo bromoalkane 10 as colorless oil (196.53 g, 98%).

¹H NMR (CDCl₃) δ: 0.85-0.21 (14H, m), 3.85-3.95 (1H, m).

Step 3. To a suspension of tBuOK (75.74 g, 0.675 mol) in dry tBuOH (400 mL) was added dropwise with stirring a solution of C-exo bromoalkane 10 (96.81 g, 0.45 mol) in dry THF (100 mL) at room temperature. The mixture was refluxed overnight under argon. After cooling, the reaction mixture was diluted with water (800 mL) and extracted with hexane. The combined organic layers were dried over anhydrous MgSO₄, filtered, and concentrated. The residual oil was distilled at 58-63° C. under vacuum (15 mmHg) to provide C-exo alkene 5 as colorless oil (38.62, 64%).

IR (neat) cm⁻¹: 2949, 1471, 1456, 1325, 693; MS (APCI) m/z: 135 (M+1); ¹H NMR (CDCl₃) δ: 0.90-1.05 (2H, m), 1.30-2.00 (8H, m), 2.40-2.50 (2H, m), 6.05-6.15 (2H, m).

Step 4. A mixture of tris-(dibenzylideneacetone)-dipalladium(0) (Pd₂ dba₃) and CHCl₃ adduct (41 mg, 0.04 mmol), R-(+)-MOP (74 mg, 0.16 mmol), and C-exo alkene 5 was sonicated for 5 min, followed by the dropwise addition of trichlorosilane (1 mL, 9.6 mmol) with stirring under argon at bath temperature of 0° C. The specific chiral monodentate phosphine ligand of Formula II used in this reaction has R configuration with R⁵ as —OCH₃, and R⁶ and R⁷ as phenyl. The mixture was then stirred at the same temperature overnight. The reaction mixture was diluted by adding hexane dropwise, filtered, and washed with hexane. The combined organics were concentrated to give crude optically active Si-exo/C-exo organosilane 6 as colorless oil, which was used directly in the next step without further purification.

Step 5. To a mixture of crude Si-exo/C-exo organosilane 6, KHCO₃ (5.82 g), and KF (2.25 g) in THF (10 mL) and MeOH (10 mL), cooled with an ice bath, was added 30% aqueous H₂O₂ (5.1 mL) dropwise with stirring. After stirred overnight, the reaction mixture was extracted with CHCl₃. The combined organic layers were washed with water, dried over anhydrous Mg₂SO₄, filtered, and concentrated. The crude product was purified with silica chromatograph to provide optically active (−)-O-exo/C-exo alkanol 7 as colorless oil (954 mg, 79%).

IR (neat) cm⁻¹: 3347, 2941, 2861; MS (APCI) m/z: 135 (M+1−H₂O); ¹H NMR (CDCl₃) δ: 0.85-1.05 (2H, m), 1.15-1.40 (5H, m), 1.50-2.00 (8H, m), 3.70-3.75 (1H, m).

The enantiomeric excess (e.e.) and exo/endo ratio of optically active (−)-O-exo/C-exo alkanol 7 was determined by converting the molecule into a carbamate as follows.

To a solution of optically active (−)-O-exo/C-exo alkanol 7 (91 mg, 0.6 mmol) in THF (5 mL) was added 3,5-dinitrophenyl isocyanate (150 mg, 0.72 mmol). The reaction mixture was stirred at room temperature for 3 hrs. After removing the solvent, the residue was purified with silica chromatography to yield the corresponding carbamate as pale yellow amorphous (84 mg).

HPLC (chemical purity): 99.2%; HPLC (optical purity): 83% e.e.; MS (ESI) m/z: 360 (M−1); Exo/endo ratio: greater than 99%.

The asymmetric hydrosilylation reaction was also optimized by varying the catalyst and other reaction conditions. The results are summarized in Table 1.

TABLE 1
The Optimization of Asymmetric Hydrosilylation

| | | Temperature | Yield (%) | Optical Purity |
|---|---|---|---|---|
| Catalyst | mol % | (° C.) (5→6) | (5→7) | of 7 (% e.e.) |
| [PdCl(π-C₃H₅)]₂ | 5 | 0 | 15 | 75 |
| Pd₂dba₃•CHCl₃ | 1 | 0 | 26 | 85 |
| Pd₂dba₃•CHCl₃ | 1 | 0 | 79^{a} | 83 |
| Pd₂dba₃•CHCl₃ | 1 | 0 | 31^{b} | 84 |
| Pd₂dba₃•CHCl₃ | 1 | 0 | 27^{c} | 80 |
| Pd₂dba₃•CHCl₃ | 1 | 20^{d} | 14^{a} | 58 |
| Pd₂dba₃•CHCl₃ | 1 | −20 | 81^{a} | 87 |
| Pd₂dba₃•CHCl₃ | 1 | −40 | 6^{a} | 74 |
| Pd₂dba₃•CHCl₃ | 0.5 | 0 | 16^{a} | 60 |

^{a}Prior to the addition of trichlorosilane, a mixture of Pd₂dba₃•CHCl₃ adduct, (R)-MOP, and compound 5 was sonicated for 5 min.
^{b}Prior to the addition of compound 5, a mixture of Pd₂dba₃•CHCl₃ adduct, (R)-MOP, and trichlorosilane was sonicated for 5 min.
^{c}Prior to the addition of compound 5 and trichlorosilane, a mixture of Pd₂dba₃•CHCl₃ adduct and (R)-MOP in CHCl₃ was sonicated for 5 min and the solvent was removed.
^{d}After the addition of trichlorosilane, the inner temperature rose to more than the boiling point of trichlorosilane.

Step 6. To a solution of optically active (−)-O-exo/C-exo alkanol 7 (79.5 mg, 0.52 mmol, 78% e.e.) in dry THF (2 mL), cooled with an ice bath, was added tBuOK (53 mg, 0.47 mmol) with stirring, followed by the dropwise addition of a solution of CS₂ (40 mg, 0.53 mmol) in dry THF (3 mL). The mixture was stirred overnight at room temperature. After removing the solvent, the residue was triturated with Et₂O and dried to yield optically active potassium salt of (−)-O-exo/C-exo-tricyclo[5.2.1.0^{2,6}]-dec-9-yl-xanthic acid 1A as pale yellow solid (99 mg, 71%).

MS (ESI) m/s: 227 (M-K); ¹H NMR (CD₃OD) δ: 0.80-1.10 (2H, m), 1.10-2.10 (11H, m), 2.15-2.20 (1H, m), 5.05-5.10 (1H, m); [α]D₂₀: −12.5° (c 1.04, H₂O).

### Example 2

### Synthesis of optically active O-exo/C-exo-tricyclo[5.2.1.0^{2,6}]-dec-9-yl-xanthic acid 1A

The syntheses of optically active potassium salt of (+)- and (−)-O-exo/C-exo-tricyclo[5.2.1.0^{2,6}]-dec-9-yl-xanthic acid 1A are illustrated in Schemes 6 to 8.

Step 1. Cyclopentadiene 8 (50 g) in H₂SO₄ (25% by weight, 150 mL) was stirred mechanically under nitrogen at 107° C. for 5 hrs. After cooled to room temperature, the reaction mixture was separated into an aqueous and organic layer. The organic layer was separated from the aqueous layer, washed with water, diluted with t-butyl methyl ether (250 mL), and concentrated in vacuo to provide alkenol 12 as colorless oil (55 g, 100%).

Step 2. A mixture of alkenol 12 (200 g) and Pd/C (3.75% by weight, 7.5 g) in ethanol (600 mL) was charged to an autoclave. The mixture was stirred at room temperature overnight under hydrogen (5 bars). The reaction was monitored by ¹H NMR. After the reaction was completed, the reaction mixture was filtered through Celite (400 g) and concentrated in vacuo to provide alkanol 7 as colorless oil (200 g, 100%).

Step 3. To a solution of alkanol 7 (2 g) in pyridine (8 mL) were added acetic anhydride (1.35 mL) and dimethylaminopyridine (290 mg) under nitrogen. The mixture was stirred at 50° C. for 3.5 hrs. After neutralized with hydrochloric acid (2 M, 30 mL), the reaction mixture was extracted with CH₂Cl₂. The combined organic layers were dried over anhydrous Na₂SO₄ and concentrated in vacuo to provide ester 13 as pale yellow oil (2 g).

Step 4. Ester 13 (20 mg) in a mixture of t-butyl methyl ether (0.2 mL) and 0.1 M KH₂PO₄ buffer solution at pH 7 (1 mL) in the presence of one of the three enzymes (2 mg) listed in Table 2 was agitated on a shaker overnight. All three enzymes were obtained from Mann Associates (London, UK). These enzymes selectively hydrolyze (+) ester 13, thus producing optically active (+) alkanol 7 and (−) ester 13. Their optical purities were analyzed by chiral GC as described by Holscher et al. (Helv. Chim. Acta 2004, 87, 1666-1680), and the results are summarized in Table 2.

TABLE 2
Enzymatic Resolution of Ester 13.

| Enzyme | Source | Optical Purity |
|---|---|---|
| Peptidase | Rhizopus oryzae | 92% e.e. |
| Lipase | Pseudomonas fluorecens | 40% e.e. |
| Lipase A | Candida Antarctica | 40% e.e. |

Step 5. (−)-Ester 13 (25 g) was dissolved in methanol (150 mL). An aqueous NaOH solution (4 M, 65 mL) was added and the mixture stirred at 22° C. for 60 min. The reaction mixture was concentrated under reduced pressure and the residue was partitioned between water (100 mL) and methyl tert-butyl ether (100 mL). The aqueous layer was extracted with methyl tert-butyl ether (100 mL) and the combined organic extracts were dried over Na₂SO₄. The solvent was removed under reduced pressure to yield (−) alkanol 7 (19.9 g).

Step 6. To a solution of sodium t-butoxide (1.6 g) in THF (10 mL) was added optically active (−)-alkanol 7, followed by dropwise addition of carbon disulfide (1.5 g). The reaction mixture was then held at room temperature overnight. The reaction mixture was filtered and washed with diethylether to provide optically active potassium salt of (−)-O-exo/C-exo-tricyclo[5.2.1.0^{2,6}]-dec-9-yl-xanthic acid 1A.

Using the same procedure, optically active (+)-alkanol 7 was also converted to optically active potassium salt of (+)-O-exo/C-exo-tricyclo[5.2.1.0^{2,6}]-dec-9-yl-xanthic acid 1A.

### Example 3

### Synthesis of optically active O-exo/C-exo-tricyclo[5.2.1.0^{2,6}]-dec-9-yl-xanthic acid 1A

The syntheses of optically active potassium salt of (+)- and (−)-O-exo/C-exo-tricyclo[5.2.1.0^{2,6}]-dec-9-yl-xanthic acid 1A are illustrated in Schemes 6 and 7.

Step 1. To a solution of alkenol 12 (2 g) in pyridine (8 mL) were added acetic anhydride (1.35 mL) and dimethylaminopyridine (290 mg) under nitrogen. The mixture was stirred at 50° C. for 3.5 hrs. After neutralized with hydrochloric acid (2 M, 30 mL), the reaction mixture was extracted with CH₂Cl₂. The combined organic layers were dried over anhydrous Na₂SO₄ and concentrated in vacuo to provide unsaturated ester 11 as pale yellow oil (2 g).

Step 2. Ester 11 (20 mg) in a mixture of t-butyl methyl ether (0.2 mL) and 0.1 M KH₂PO₄ buffer solution at pH 7 (1 mL) in the presence of one of the hydrolytic enzymes (2 mg) listed in Table 2 was agitated on a shaker overnight. All three enzymes were obtained from Mann Associates (London, UK). These enzymes selectively hydrolyze (+)-ester 11, thus producing optically active (+) alkenol 12 and optically active (−) ester 11. Their optical purities were analyzed by chiral GC as described by Holscher et al. (Helv. Chim. Acta 2004, 87, 1666-1680), and the results are summarized in Table 3.

Step 3. Potassium carbonate (21.6 g) was added to a solution of (−) ester 11 (10 g) in methanol (50 mL) under nitrogen. The mixture was stirred at room temperature overnight and monitored by TLC (CH₂Cl₂, PMA stain) until disappearance of acetate. Water (30 mL) and methyl tert-butyl ether (30 mL) were added. The aqueous layer was extracted with methyl tert-butyl ether (3×20 mL) and the combined organic layers were dried over Na2SO4 and evaporated to dryness to give (−) alkenol 12 (7.6 g, 98%).

Step 4. A solution of (−) 12 (7.6 g) in ethanol (40 mL) was charged into an autoclave. Pd/C (3.75% w/w, 285 mg) was added and the mixture was stirred at room temperature overnight under H₂ (5 bars). The reaction was monitored by ¹H NMR until completion, then filtered through Celite (5 g), and concentrated to give (−) 7 (7.8 g) as a dark oil.

Step 5. Optically active (+)- and (−)-alkanols 7 were converted into optically active potassium salt of (+)- and (−)-O-exo/C-exo-tricyclo[5.2.1.0^{2,6}]-dec-9-yl-xanthic acid 1A, using the procedure described herein.

TABLE 3
Enzymatic Resolution of Unsaturated Ester 11.

| Enzyme | Source | Optical Purity |
|---|---|---|
| Peptidase | Rhizopus oryzae | 100% e.e. |
| Lipase B | Candida Antarctica | 100% e.e. |
| Lipase B (Immobilized) | Candida Antarctica | 100% e.e. |

### Example 4

### Synthesis of optically active O-exo/C-exo-tricyclo[5.2.1.0^{2,6}]-dec-9-yl-xanthic acid 1A

Step 1. Synthesis of alcohol +/−12. Cyclopentadiene 8 (50 g) was heated in 25% w/w H₂SO₄ (150 mL) at 107° C. for 5 hrs under nitrogen. The reaction was cooled and the layers were separated. Organic layer was washed with water and diluted with tert-butyl methyl ether (250 mL). The tert-butyl methyl ether was then reduced in vacuo to provide alcohol +/−12 (55 g, 100%).

Step 2. Synthesis of acetate +/−11 (R⁹=Me). To alkenol +/−12 (537 g) were added acetic anhydride (340 mL), triethylamine (490 mL) and N-methylimidazole (2.5 mL). The mixture was stirred at 50° C. for 2.5 hrs, and then tent-butyl methyl ether (540 mL) and 2M HCl (490 mL) were added. The layers were separated and the aqueous layer was further extracted twice with tert-butyl methyl ether (540 mL). Combined organic phases were washed with 5% NaHCO₃ (270 mL), brine (540 mL), and concentrated to give acetate +/−11 (R⁹=Me) (590 g, 96%).

Step 3. Synthesis of acetate +/−/3 (R⁹=Me). Acetate+/−11 (R⁹=Me) (600 g) in ethanol (1500 mL) was hydrogenated at 3-bar using Pd/C (20 g) for 2 hrs at 25° C. On completion (as judged by GC), the reaction medium was filtered through Celite and concentrated to yield acetate +/−13 (R⁹=Me) (606 g).

Step 4. Bioresolution of acetate +/−/3 (R⁹=Me). Potassium phosphate dibasic (82.5 g) was added to water (6 L), stirred for 30 min, and then the pH was adjusted to pH 7 with 2M NaOH. The solution was warmed to 35° C. and +/−13 (R⁹=Me) (500 g) was added in one portion, followed by enzyme AE015 (300 g) and 0.1M potassium phosphate dibasic buffer (1 L). On completion of the reaction, as judged by GC, sodium chloride (50 g) and toluene (2.5 L) were added. The mixture was stirred for 5 min, allowed to separate and the aqueous extracted into toluene (2×2.5 L). Combined organics were washed with brine (2.5 L) then filtered through Celite (400 g). Concentration of the organic layers yielded crude material (480 g) containing acetate—13 (R⁹=Me) (96 g) and alkanol +7 (197 g).

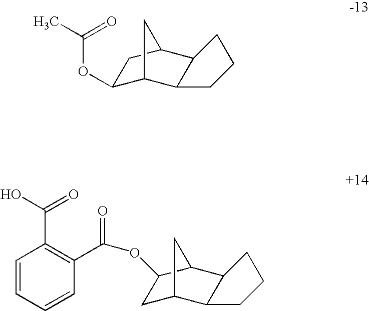

Step 5. Phthalate +14 formation and isolation of acetate—13 (R⁹=Me). To pyridine (107 mL) was added the mixture (107 g) of unwanted alcohol +7 and desired acetate—13 (R⁹=Me) from the bioresolution step. Phthalic anhydride (65 g) and N,N-dimethylaminopyridine (4.3 g) were then added and the reaction heated at 60° C. for 5 hrs. On cooling to 10° C., 2M HCl (214 mL) was added dropwise maintaining the internal temperature below 20° C. Tert-butyl methyl ether (214 mL) was added and stirred for 5 min. Organic layer was separated and washed with 2M HCl (214 mL). Combined aqueous fractions were extracted into tert-butyl methyl ether (214 mL). Organic fractions were washed with 1M NaOH (2×214 mL), brine (214 mL), and concentrated to dryness to yield acetate—13 (R⁹=Me) (49.1 g).

Step 6. Synthesis of alkanol—7 containing isomeric impurity. Sodium hydroxide (11.8 g) was added to acetate—13 (R⁹=Me) (52 g) in methanol (260 mL) under nitrogen atmosphere. The reaction was stirred at 25° C. for 2 hrs, methanol was distilled out of the reaction, and then tert-butyl methyl ether (75 mL) and water (75 mL) were added. The mixture was stirred for 5 min. The layers were separated and further water (2×75 mL) was added. Brine (75 mL) was added and the mixture was stirred for 5 min, and then ammonium chloride (75 mL) was added and stirred for 5 min. The layers were separated and organic layer was concentrated to yield alkanol—7 (37.1 g).

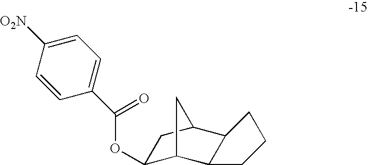

Step 7. Synthesis of p-nitrobenzoate—15. Alkanol—7 (39.8 g, contaminated by-products and isomers) was dissolved in 130 mL pyridine (130 mL). p-Nitrobenzoylchloride (231 g) 25% w/w solution in dichloromethane was added dropwise while cooling the mixture in an ice bath. Stirring was continued at 22° C. for 18 hrs. After addition of water (130 mL), the mixture was stirred for 60 min. Methyl tert-butyl ether (1 L) was added and the mixture washed with 2M aqueous HCl (1 L). The organic phase was washed with NaHCO₃ (aq.) and brine, and dried (Na₂SO₄). Evaporation of solvent under reduced pressure furnished the crude p-nitrobenzoate ester—15 (84.4 g) as a brown solid. Crude p-nitrobenzoate ester—15 (84.4 g) was heated in isopropanol (400 mL) to reflux. The mixture was cooled to 22° C. within 5 hrs, and seeded at 45° C. After additional 2 hrs at 22° C., the mixture was filtered. The wet cake was washed with isopropanol (50 mL) and then hexane (50 mL). After air-drying, chemically, diastereo- and enantiopure—15 (43.4 g) was obtained. A second crop of pure—15 (13.1 g) was obtained by recrystallizing the mother liquor residue again from isopropanol (190 mL) in the same manner.

Step 8. Synthesis of pure—7. Purified p-nitrobenzoate ester—15 (142 g) was heated in methanol (1.4 L) to 60° C. 4M aqueous NaOH (360 mL) was added and the mixture was stirred for 60 min at 22° C. Then most of the methanol was distilled off under reduced pressure. The residue was partitioned between water (250 mL) and methyl tert-butyl ether (250 mL). The aqueous layer was extracted with methyl tert-butyl ether (150 mL) once more. The combined organic extracts were dried over Na₂SO₄. The solvent was removed under reduced pressure to yield alkanol—7 (57.4 g) as colorless oil.

Step 9. Optically active (+)- and (−)-alkanols 7 were converted into optically active potassium salt of (+)- and (−)-O-exo/C-exo-tricyclo[5.2.1.0^{2,6}]-dec-9-yl-xanthic acid 1A, using the procedure described herein.

### Example 5

### Inhibition of Phosphatidylcholine-Specific Phospholipase C

The inhibitory activities of optically active potassium salts of (+)- and (−)-O-exo/C-exo-tricyclo [5.2.1.0^{2,6}]-dec-9-yl-xanthic acid 1A ((+)- and (−)-enantiomers 1A) against phosphatidylcholine-specific phospholipase C were evaluated, together with D609 and potassium salt of racemic O-exo/C-exo xanthic acid 1A (racemic 1A), using Amplex Red phosphatidylcholine-specific phospholipase C(PC-PLC) assay kit, which was obtained from Invitrogen (Calsbad, Calif.). D609 was obtained from Sigma Aldrich.

An Amplex Red stock solution (˜20 mM), a working reaction buffer, and a horseradish peroxidase stock solution (200 U/mL), a H₂O₂ working solution (20 mM), a choline oxidate stock solution (20 U/mL), an alkaline phosphatase stock solution (400 U/mL), and a B. cereus PC-PLC stock solution (10 U/mL) were prepared according to the instruction of the assay kit. The stock solution (100 mg/mL) for each test compound was also prepared by dissolving the compound in water 2 hr prior to the start of the experiment.

A working Amplex Red/HRP/lecithin solution was prepared by adding 200 μL of Amplex Red reagent stock solution, 100 μL of HRP stock solution, 200 μL of alkaline phosphatase stock solution, 100 μL of choline oxidase stock solution, and 78 μL of the lecithin solution to 9.32 μL of the working reaction buffer. PC-PLC solution (0.2 U/mL) was prepared by diluting the PC-PLC stock solution with the working reaction buffer to the desired concentration.

The test compound (25 μL) at a serious of concentrations was pipetted into a 96 well plate. For uninhibited PC-PLC controls and non-PC-PLC controls, 25 μL of water was added. After the addition of 50 μL of the Amplex Red/HRP/lecithin working solution, the reaction was initiated by adding 25 μL of PC-PLC solution to each well. To the non-PC-PLC controls, 25 μL of water was added instead of PC-PLC solution. The experiment was performed in triplicate. The reactions were protected from light. After incubated for 30 min at 37° C., the reactions were measured with a fluorescence microplate reader using excitation at 550 nm and emission detection at 590 nm. The fluorescent data obtained were corrected for background fluorescence by subtracting the values obtained from the non-PC-PLC controls. The IC₅₀ values were then calculated for each test compound and the results are summarized in Table 4.

TABLE 4
Inhibition of PC-Phospholipase C

| Compound | IC₅₀ (μg/mL) |
|---|---|
| D609 | 20.49 ± 1.99 |
| Racemic 1A | 10.05 ± 0.77 |
| (+)-Enantiomer 1A | 13.88 ± 0.85 |
| (−)-Enantiomer 1A | 3.62 ± 0.17 |

### Example 6

### Inhibition of Bovine Papilloma Virus (BPV)

The inhibitory activities of optically active (+)- and (−)-enantiomers 1A against bovine papilloma virus were evaluated along with D609 and potassium salt of racemic 1A using in vitro BPV-infected hamster embryo fibroblasts (HEF) cell proliferation assay, as described (Amtmann et al., Exp. Cell. Res. 1985, 161, 541-550). The results are summarized in Table 5.

Briefly, hamster embryo fibroblasts and bovine papilloma virus type I (BPV-1)-transformed HEF (HEF-BPV) were grown in Eagle's basal medium. BPB-HEP and HEF cells were seeded in 96-well plates at a density of 0.5×10⁶ cells per plate in DMEM cell culture medium containing 10% fetal calf serum at pH 6.8. The plates were then incubated in the presence of 5% CO₂ in 100% humidity at 37° C. After 6 hr, the cell culture medium in the plates was exchanged with fresh DMEM cell culture medium containing test compounds at a series of concentrations. The plates were incubated at 37° C. for additional 72 hr. Cells in the plates were then washed with PBS, fixed with 3% formaldehyde solution containing 0.9% NaCl for 1 min, washed by water for 10 sec, and dried overnight. For detection, the plates were stained with crystal violet solution for 5 min at room temperature, washed 5 times with water, and dried at room temperature overnight. After adding ethanol/acetic acid (99:1, v/v, 100 mL) to each well, the plates were measured at 595 nm using an ELISA reader.

TABLE 5
Inhibition of Bovine Papilloma Virus

| Compound | IC₅₀ (μg/mL) |
|---|---|
| D609 | 21.64 ± 1.85 |
| Racemic 1A | 14.55 ± 0.07 |
| (+)-Enantiomer 1A | 19.89 ± 2.52 |
| (−)-Enantiomer 1A | 11.07 ± 0.18 |

### Example 7

### Inhibition of Human Papilloma Virus (HPV)

The inhibitory activities of optically active (−)-enantiomer 1A against human papilloma virus were evaluated using HPV-31-infected CIN612 9E keratinocytes (Meyers et al., Science 1992, 257, 971-973).

a. Short-Term Study

Effect on cell proliferation. HPV-31 infected cells were seeded in 96-well plates containing 1×10⁶ mitomycin C treated 3T3 cells (1×10⁴ cells/well), at a density of 0.5×10⁶ cells per plate (0.5×10⁴ cells/well) in E medium. After the plates were incubated in a CO₂ incubator under 5% CO₂ at 37° C. in 100% humidity for six hrs, the cell culture medium was removed and fresh E medium containing 0.85 g/L NaHCO₃ and a test compound at a series of concentrations (0, 0.5, 1, 2, 4, 8, 16, 32, 64, and 128 μg/mL), or positive control (interferon γ at 200 IU/mL) was added. A stock solution of optically active (−)-enantiomer 1A at 100 mg/mL in double distilled H₂O was prepared not earlier than 1 hr before the assay and kept on ice prior to use. All samples were tested in quadruplicates at each concentration. One 96 well plate with untreated cells was fixed at the same time point as the cells treated with test compound or control. The plates were incubated for 72 hrs at 37° C. in a CO₂ incubator. After the cell culture medium was removed by decanting, fibroblast feeders were removed by washing twice with a solution (100 μL/well) containing 0.5 mM EDTA in PBS, and with PBS (100 μL/well). Formaldehyde (3%, 100 μL/well) was added to the plates. After 5 min, the formaldehyde was decanted and the plates were dried upside down on blotting paper overnight at room temperature. The cells were stained by adding 0.1 mL of crystal violet solution to each well. After incubation for 5 min at room temperature, the crystal violet solution was decanted and the plates were washed 5 times by submersion in 3 L fresh water. The crystal violet solution used was prepared by first dissolving crystal violet in ethanol to a concentration of 10% and then diluting the ethanol solution with double distilled H₂O (1:20). After the plates were dried on blotting paper upside down overnight, 0.1 mL of ethanol/acetic acid (99:1) was added to each well and optical density at 595 nm was determined in an ELISA reader.

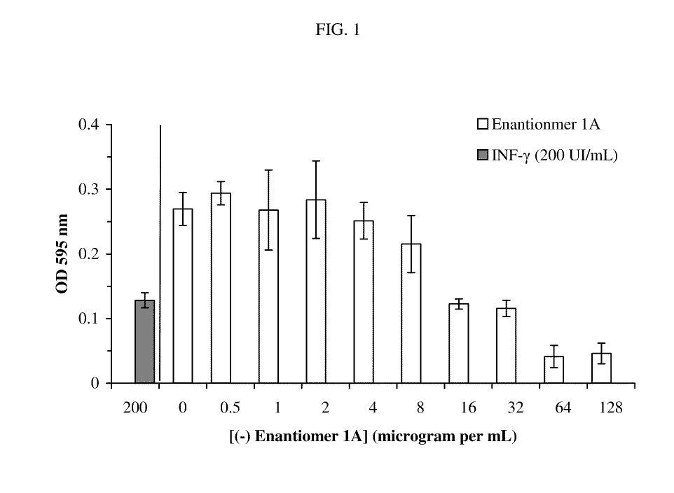

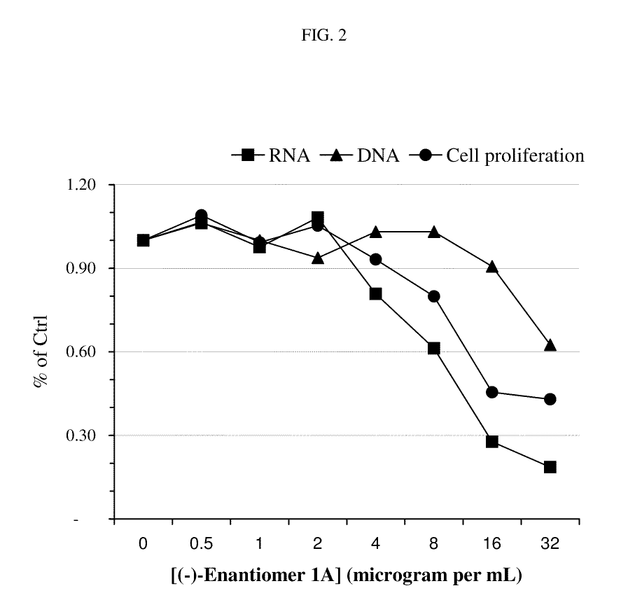

Dose response curves were established by plotting growth against concentrations of the test compound or control. The results are shown in FIGS. 1 and 2. ICₛₒ values were determined from the dose response curves from crossing points of the dose response curves with a line parallel to the x-axis corresponding to 50% of uninhibited growth. Optically active (−)-enantiomer 1A exhibited an EC₅₀ of 16 μg/mL, whereas the positive control (INF-γ) showed about 50% inhibition of the growth of HPV-31-infected CIN612 9E keratinocytes at 200 UI/mL.

Effect on HPV-31 specific DNA and RNA. HPV-31 infected cells (3×10⁶) were dispensed into 14.5 cm dishes containing fresh E culture medium and 1×10⁶ mitomycin C-treated J2 3T3 fibroblast feeders. After 6 hrs, fresh medium containing 0.85 g/L NaHCO₃ and a test compound at a series of concentrations (0, 0.5, 1, 2, 4, 8, 16, and 32 μg/mL) were added. All samples were tested in duplicates at each concentration. After incubation at 37° C. for 72 hrs, the feeder cells were removed with 4 mM EDTA, and DNA and RNA was then isolated. The isolated DNA and RNA were analyzed in Southern and Northern Blot gels for HPV-31 specific sequences. Human A431 epithelial cancer cells were used as HPV-uninfected negative control. X-ray films were scanned and optical densities of HPV-31 specific DNA and major mRNA species were integrated.

Dose response curves were established by plotting integrated values against the concentrations of the test compound. The results are shown in FIG. 2. The IC₅₀ values were determined from the dose response curves from crossing points of the dose response curves with a line parallel to the x-axis corresponding to 50% of uninhibited integrals. Optically active (−)-enantiomer 1A exhibited an IC₅₀ of 10.7 μg/mL on the inhibition of HPV-31 specific RNA expression. The expression of the control RNA (actin specific RNA) was only affected at the highest concentration of 32 μg/mL of optically active (−)-enantiomer 1A. On the inhibition of HPV-31 specific DNA, optically active (−)-enantiomer 1A showed 37.5% inhibition at 32 μg/mL.

b. Long-Term Study

HPV-31 infected cells (3×10⁶) were dispensed into 14.5 cm dishes containing fresh E medium and 1×10⁶ cells of mitomycin C-treated 3T3 fibroblast feeders. The dishes were incubated in a CO₂ incubator under 5% CO₂ at 37° C. in 100% humidity. Human A431 epithelial cancer cells were used as HPV-uninfected negative controls. After 6 hr incubation, the cell culture medium was removed and fresh E medium containing 0.85 g/L NaHCO₃ and a test compound at concentrations of 10 and 3.3 μg/mL, or control (interferon γ at 200 IU/mL) were added. For each concentration, 4 dishes were prepared. The dishes were incubated at 37° C. in a CO₂ incubator. The culture medium was replaced to new E medium containing each concentration of the test compound or control in every 72 hrs. After seven days, or when untreated cells became confluent, one dish of each group was harvested for DNA/RNA extraction, followed by Southern/Northern Blot analysis; and one dish was trypsinized, cell number were determined and cells were seeded in three new dishes, treated and cultured as described above. This procedure was repeated for 9 passages. During these passages, morphology of cells and cell density were monitored.

Effect on cell proliferation. Cell numbers in the dishes harvested were determined in a Neubauer hematocytometer. The multiplication factor was determined at every step of passage. The cell number of each culture at the end of the passage was divided by the number of seeded cells at the start of each passage. The end point was reached when keratinocytes treated with a test compound stopped growing. Stop of cell growth was defined as a multiplication factor of no greater than 1.

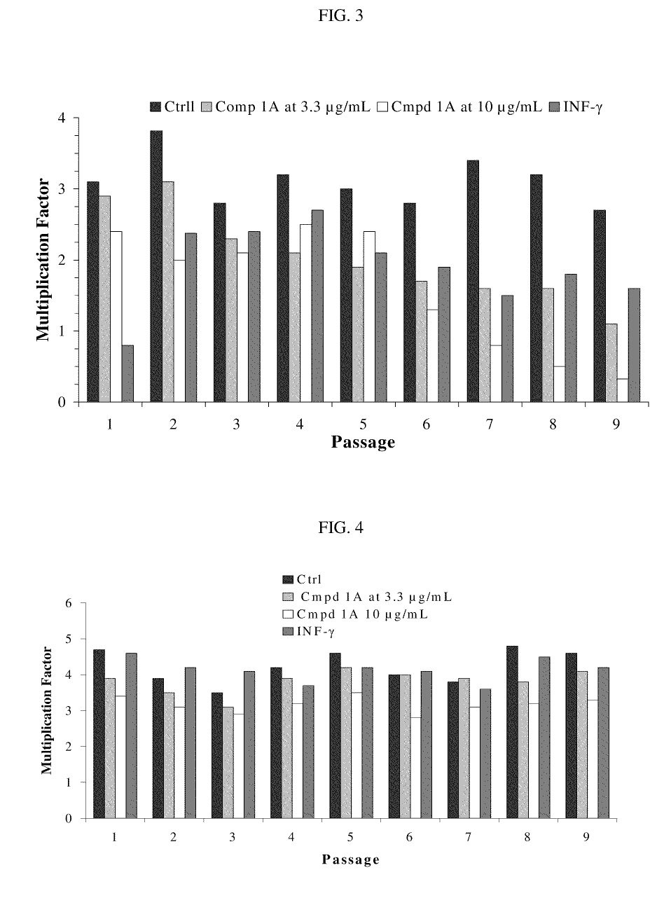

The growth of untreated CIN612 9E cells was rather constant throughout the 9 passages. The factor of multiplication varied between 2.7 and 3.9. There was no obvious loss of viability during the observation period. In contrast, treatment with all test compounds affected cell growth (FIG. 3).

Treatment with INF-γresulted in a high rate of cell death during the first cell passage, leading to a multiplication factor below 1 (0.8). However, the multiplication factor increased sharply in the second passage (2.4), remained at that level for further 3 passages and somewhat decreased to a value between 1.9 and 1.5 during the last four passages. However, the decrease was not cumulative.

Treatment of CIN612 9E cells with optically active (−)-enantiomer 1A at a concentration of 3.3 μg/mL resulted in a reduction of the growth rate. The multiplication factor decreased from 2.9 (passage 1) to 1.1 (passage 9), that is, the cells almost stopped growing after 9 passages.

Treatment of CIN612 9E cells with optically active (−)-enantiomer 1A at a concentration of 10 μg/mL resulted in a significant reduction of cell proliferation. The multiplication factor decreased from 3 for untreated cells to a value of 2-2.5 during the first five passages. After passage 5, a steep and cumulative reduction of the multiplication factor was observed (passage 5: 2.4, passage 6: 1.3, passage 9: 0.1). From passage 7 on, the multiplication factor was <1, indicating that cells were dying.

In comparison, the growth of control A431 cells (epithelial cancer cells) was rather constant or reduced small extent compared with those of CIN 9E cells throughout the 9 passages (FIG. 4). The factor of multiplication of control A431 cell untreated with neither optically active (−)-enantiomer 1A nor IFN-γ were between 3.5 and 4.7. Treatment of A431 cells with 200 IU/mL of INF-γhad no significant effect on cell growth. The factor of multiplication was between 3.7 and 4.6 throughout the 9 passages. Treatment of A431 cells with optically active (−)-enantiomer 1A had only minor effects on cell growth. Treatment with optically active (−)-enantiomer 1A at a concentration of 3.3 μg/mL resulted in slight inhibition of cell growth. The factor of multiplication varied between 3.1 and 4.2. There was no cumulative effect. Treatment with optically active (−)-enantiomer 1A at a concentration of 10 μg/mL also resulted in slight inhibition of cell growth. The factor of multiplication varied between 2.8 and 3.5. There was also no cumulative effect.

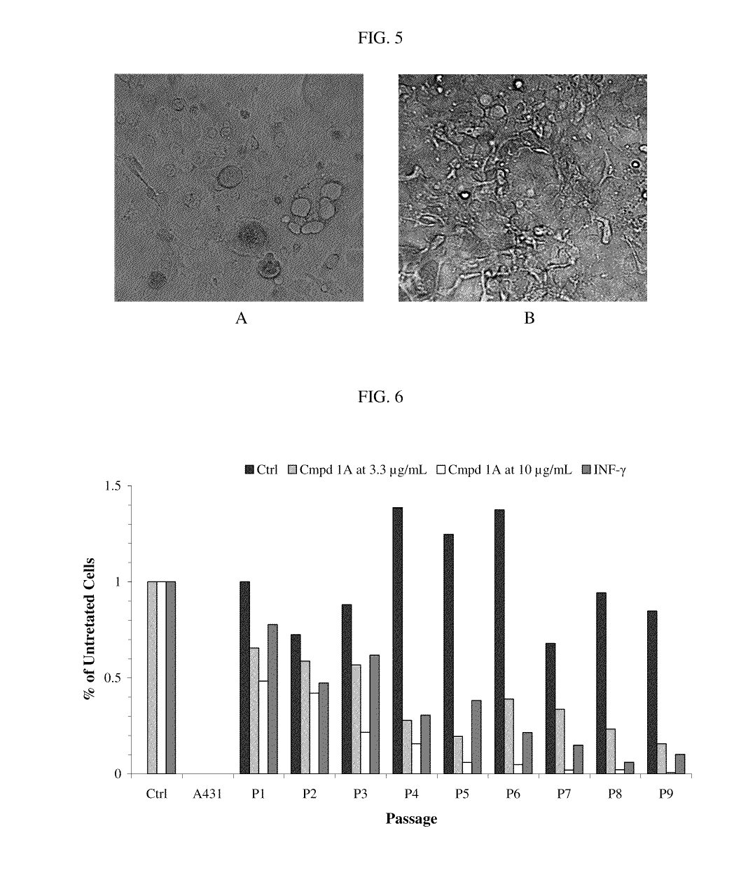

After five passages, a notable change in the morphology of CIN 612 9E cells treated with 10 μg/mL of optically active (−)-enantiomer 1A was observed (FIGS. 5A and 5B). Cells were growing more organized like untransformed keratinocytes, instead of the criss-cross pattern of untreated CIN 612 9E cells. CIN 612 9E cells treated with optically active (−)-enantiomer 1A were contact inhibited when cultures grew to confluency, while untreated CIN 612 9E cells were piling up and did not stop their growth after reaching confluency. In addition, CIN 612 9E cells treated with optically active (−)-enantiomer 1A acquired a flat round shape, whereas untreated CIN 612 9E cells kept the spindle form.

Effect on HPV-31 specific DNA and RNA: DNA and RNA were analyzed in Southern and Northern Blot gels for HPV-31 specific sequences. X-ray films were scanned and optical densities of HPV-31 specific DNA and major mRNA species were integrated. Time curves were established by plotting integrated values against number of passages for each treatment dose. The T₅₀ (time required to reduce the level to 50% of control) was determined from the time curves from crossing points of time curves with a line parallel to the x-axis corresponding to 50% of integrals of untreated control sample.

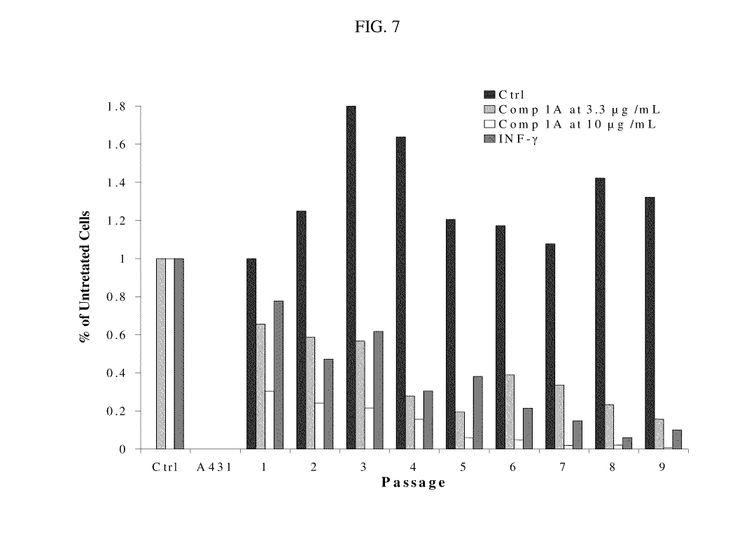

The results are shown in FIGS. 6 and 7, and summarized in Table 6. The cells treated with optically active (−)-enantiomer 1A at 3.3 μg/mL or 10 μg/mL or with INF-γ at 200 IU/mL all showed continuous reductions of HPV-31 specific DNA and RNA contents (Table 6). The most effective treatment was with 10 μg/mL of optically active (−)-enantiomer 1A. The number of viral genomes per cell was reduced by more than 50% after a single passage (T₅₀ DNA <1 and T₅₀ RNA <1) and to <5% after 6 passages. After 9 passages, viral DNA and RNA were almost undetectable (below 1%).

TABLE 6
Inhibition of HPV-31 specific DNA and RNA expression

| Treatment | T₅₀ DNA¹ | T₅₀ RNA² |
|---|---|---|
| Control (No cmpd or Ctrl) | No effect | No effect |
| Cmpd 1A at 10 μg/mL | <1 | <1 |
| Cmpd 1A at 3.3 μg/mL | 2.23 | 3.28 |
| INF-γ at 200 IU/mL (Ctrl) | 2.7 | 3.36 |

¹Number of passages required to reduce the amount of HPV-31 specific RNA by 50%
²Number of passages required to reduce the amount of HPV-31 specific DNA by 50%

### Example 8

### Inhibition of Herpes Simplex Virus Type-2 (HSV-2)

The inhibitory activities and cytotoxicity of potassium salts of optically active (+)- and (−)-enantiomers 1A against herpes simplex virus type 2 were evaluated along with D609, potassium salt of racemic 1A, and aciclovir. The results are summarized in Table 6.

Calu-6 cells and RITA cells were grown in DMEM cell culture medium containing 10% fetal calf serum in CO₂ incubator under 5% CO₂ at 37° C. with 100% humidity.

For cytotoxicity assay, Calu-6 cells were seeded in 96 well plates at a density of 3×10⁶ cells per plate in the DMEM cell culture medium. The plates were incubated at 37° C. in the presence of 5% CO₂ for 24 hr. The DMEM cell culture medium in the plates was exchanged with fresh DMEM cell culture medium containing test compounds at a series of concentrations. The plates were incubated at 37° C. in the presence of 5% CO₂ for 48 hr. The plates were then washed with PBS, fixed with 3% formaldehyde solution containing 0.9% NaCl for 1 min, washed by water for 10 sec, and dried overnight. For detection, the plates were stained with crystal violet solution for 5 min at room temperature, washed 5 times with water, and dried at room temperature overnight. After adding ethanol/acetic acid (99:1, v/v, 100 mL) to each well, the plates were measured at 595 nm using an ELISA reader. A dose response curve was obtained for each compound tested by plotting the mean values of the optical densities against compound concentration. The LD₅₀ values obtained from the dose responses curves are summarized in Table 5.

For HSV-2 inhibition, Calu-6 cells were seeded in 96 well plates at a density of 3×10⁶ cells per plate in the DMEM cell culture medium. The plates were incubated at 37° C. in the presence of 5% CO₂ for 24 hr. The DMEM cell culture medium in the plates was removed and the cells were infected with Herpes simplex virus type-2 at 50 plaque forming units per well. After incubation at 37° C. for 60 min, test compounds in DMEM cell culture medium at a series of concentrations were added. The plates were incubated at 37° C. in the presence of 5% CO₂ for 48 hr. The plates were frozen at −20° C. prior to further analysis. After thawing at room temperature, supernatants containing infectious virus particles were prepared by centrifuging the supernatant from each well at 18,000 g at 4° C. for 5 min. The supernatant was further diluted before assay.

RITA cells were seeded in 24 well Linbra plates at a density of 4×10⁶ cells per plate in 2 mL of DMEM cell culture medium. The plates were incubated at 37° C. in the presence of 5% CO₂ for 24 hr. After the removal of the cell culture medium, 0.1 mL of the diluted supernatants was added and the plates were incubated at 37 C for 1 hr. DMEM cell medium containing 10% fetal calf serum and 0.5% methyl cellulose was added and the plates were incubated at 37 for 48 hr. The plates were then washed with PBS, fixed with 3% formaldehyde solution containing 0.9% NaCl for 1 min, washed by water for 10 sec, and dried overnight. For detection, the plates were then stained with crystal violet solution for 5 min at room temperature, washed 5 times with water, and dried at room temperature overnight. The number of plaques was determined macroscopically. The number of plaques per well was then multiplied with the dilution factor. Dose response curves were obtained by plotting the number of plaques/0.1 mL against compound concentration. The therapeutic index was also calculated for each compound by dividing LD₅₀ with IC₅₀.

TABLE 7
Inhibition of HSV-2

| Compound | IC₅₀ (μg/mL) | LD₅₀ (μg/mL) | Therapeutic Index |
|---|---|---|---|
| D609 | 30.25 ± 6.77 | 105.18 ± 21.97 | 3.5 |
| Racemic 1A | 17.18 ± 5.95 | 96.93 ± 7.12 | 5.6 |
| (+)-Enantiomer 1A | 20.97 ± 4.06 | 81.29 ± 4.53 | 3.9 |
| (−)-Enantiomer 1A | 9.08 ± 3.40 | 94.32 ± 7.33 | 10.4 |
| Aciclovir | 1.21 ± 0.81 | 65.45 ± 6.21 | 54.1 |

### Example 9

### Topical Formulation

Ointment:

| Formula | Quantity Per 100 g | |
|---|---|---|
| Active Ingredient | 0.1 g | 10 g |
| (−)-Enantiomer 1A (98% e.e.) | | |
| Vasline | 99.9 g | 90 g |

The active ingredient, (−)-O-exo/C-exo-tricyclo[5.2.1.0^{2,6}]-dec-9-yl-xanthic acid 1A, or a pharmaceutically acceptable salt or solvate thereof, and vasline are blended until uniform.

### Example 10

### Topical Formulation

Ointment:

| Formula | Quantity Per 100 g | |
|---|---|---|
| Active Ingredient | 0.1 g | 10 g |
| (−)-Enantiomer 1A (98% e.e.) | | |
| Vasline | 99.5 g | 50 g |
| Cholesterol | 0.4 g | 40 g |

The active ingredient, (−)-O-exo/C-exo-tricyclo[5.2.1.0^{2,6}]-dec-9-yl-xanthic acid 1A, or a pharmaceutically acceptable salt or solvate thereof, and vasline and cholesterol are blended until uniform.

### Example 11

### Topical Formulation

Ointment:

| Formula | Quantity Per 100 g | |
|---|---|---|
| Active Ingredients | 0.1 g | 10 g |
| (−)-Enantiomer 1A (98% e.e.) | | |
| Acicovir | 0.01 g | 5 g |
| Vasline | 99.89 g | 85 g |

The active ingredients, (−)-O-exo/C-exo-tricyclo[5.2.1.0^{2,6}]-dec-9-yl-xanthic acid 1A and acicovir, or a pharmaceutically acceptable salt or solvate thereof, and vasline are blended until uniform.

The examples set forth above are provided to give those of ordinary skill in the art with a complete disclosure and description of how to make and use the embodiments, and are not intended to limit the scope of the disclosure. Modifications of the above-described modes for carrying out the disclosure that are obvious to persons of skill in the art are intended to be within the scope of the following claims. All publications, patents, and patent applications cited in this specification are incorporated herein by reference as if each such publication, patent, or patent application were specifically and individually indicated to be incorporated herein by reference.

1. Optically active (+)-O-exo/C-exo-tricyclo[5.2.1.0^{2,6}]-dec-9-yl-xanthic acid, or pharmaceutically acceptable salt or solvate thereof.

2. The compound of claim 1, wherein the compound is a salt.

3. The compound of claim 1, wherein the compound is potassium, sodium, or lithium salt.

4. The compound of claim 1, wherein the compound is potassium salt.

5. The compound of claim 1, wherein the compound has an enantiomeric excess of no less than about 80%.

6. A pharmaceutical composition comprising the compound of claim 1, and one or more pharmaceutically acceptable excipient.

7. The pharmaceutical composition of claim 6, wherein the composition is formulated for oral, topical, or parental administration.

8. The pharmaceutical composition of claim 6, wherein the composition is formulated as a capsule or tablet.

9. The pharmaceutical composition of claim 6, wherein the composition is provided as unit dosage.

10. A method for treating a disease caused by a virus in a subject, which comprises administering to the subject the compound of claim 1.

11. The method of claim 10, wherein the disease is selected from the group consisting of molluscum contagiosum infection, HTLV infection, HTLV-1 infection, AIDS, human papillomavirus infection, herpesvirus infection, genital herpes infection, viral dysentery, flu, measles, rubella, chickenpox, mumps, polio, rabies, mononucleosis, ebola, respiratory syncytial virus infection, dengue fever, yellow fever, lassa fever, arena virus infection, bunyavirus infection, filovirus infection, flavivirus infection, hantavirus infection, rotavirus infection, viral meningitis, west Nile fever, arbovirus infection, parainfluenza, smallpox, epstein-barr virus infection, dengue hemorrhagic fever, cytomegalovirus infection, infant cytomegalic virus infection, progressive multifocal leukoencephalopathy, viral gastroenteritis, hepatitis, cold sores, ocular herpes, meningitis, encephalitis, shingles, encephalitis, california serogroup virus infection, St. Louis encephalitis, rift valley fever, hand, foot, & mouth disease, hendra virus infection, enterovirus infection, astrovirus infection, adenovirus infection, Japanese encephalitis, lymphocytic choriomeningitis, roseola infantum, sandfly fever, SARS, warts, cat scratch disease, slap-cheek syndrome, orf, pityriasis rosea, lyssavirus infection, H5N1 virus infection, and human papaloma virus infection.

12. The method of claim 10, wherein the disease is a wart, cervical dysplasia, recurrent respiratory papillomatosis, or a cancer associated with papillomavirus infection.

13. The method of claim 10, wherein the disease is cervical, anal and perianal, vulvar, vaginal, or penile cancer.

14. The method of claim 10, wherein disease is an anogenital cancer, head and neck cancer, or skin cancer.

15. The method of claim 14, wherein the head and neck cancer is oral pharyngeal region or esophagus cancer.

16. A method for inhibiting a viral infection in a subject, which comprises administering to the subject the compound of claim 1.

17. A method for inhibiting the replication of a virus, which comprises contacting the virus with the compound of claim 1.

18. The method of claim 10, wherein the virus is selected from the group consisting of adenoviruses, arbovirus, arenavirus, astroviruses, bunyaviruses, coronaviruses, Coxsackievirus, cytomegalovirus, dengue virus, ebolavirus, enteroviruses, Epstein-Barr virus, flavivirus, filoviruses, H5N1 virus, hendravirus, human T-lyphotropic viruses, human immunodeficiency viruses, human papillomaviruses, hantaviruses, hepatitis viruses, hepadnavirus, herpesviruses, herpes simplex viruses-1, herpes simplex virus-2, infant cytomegalic virus, influenza viruses, Japanese encephalitis virus, JC virus, lassa virus, lymphocytic choriomeningitis virus, lyssavirus, molluscum contagiosum virus, mumps virus, orf virus, parainfluenza viruses, paramyxovirus, parapoxvirus, parvovirus, picornavirus, poliovirus, polyomavirus, rabies virus, rift valley fever virus, Roseolovirus, rotaviruses, rubella virus, smallpox viruses, St. Louis encephalitis virus, varicella zoster virus, West Nile virus, and yellow fever virus.

19. The method of claim 10, wherein the virus is a sexually transmissible virus.

20. The method of claim 10, wherein the virus is an oncogenic virus.

21. The method of claim 10, wherein the virus is papovavirus.

22. The method of claim 10, wherein the virus is polyoma or papilloma virus.

23. The method of claim 22, wherein the virus is papilloma virus.

24. The method of claim 23, wherein the papilloma virus is human papilloma virus.

25. The method of claim 10, wherein the virus is herpes simplex virus.

26. A method for inhibiting the activity of phospholipase C, which comprises contacting phospholipase C with the compound of claim 1.

27. A method for preparing the compound of claim 1, comprising the steps of:
a) reacting achiral C-exo alkene 5 with a silane in the presence of a transition metal catalyst complexed with a chiral monodentate phosphine to produce optically active organosilane 6;
b) oxidizing the optically active organosilane 6 with an oxidant to produce optically active alkanol 7 with the retention of stereochemistry; and
c) converting the optically active alkanol 7 to optically active (−)-O-exo/C-exo-tricyclo[5.2.1.0^{2,6}]-dec-9-yl-xanthic acid 1A, or a pharmaceutically acceptable salt, solvate, or prodrug thereof.

28. A method for preparing optically active (+)-O-exo/C-exo-tricyclo[5.2.1.0^{2,6}]-dec-9-yl-xanthic acid, or a pharmaceutically acceptable salt, solvate, or prodrug thereof, which comprises the steps of:
a) reacting achiral C-exo alkene 5 with a silane in the presence of a transition metal catalyst complexed with a chiral monodentate phosphine to produce optically active organosilane 6;
b) oxidizing the optically active organosilane 6 with an oxidant to produce optically active alkanol 7; and
c) converting the optically active alkanol 7 to optically active (+)-O-exo/C-exo-tricyclo[5.2.1.0^{2,6}]-dec-9-yl-xanthic acid 1A, or a pharmaceutically acceptable salt, solvate, or prodrug thereof.

29. The method of claim 27, wherein the chiral monodentate phosphine is a compound of Formula (V): 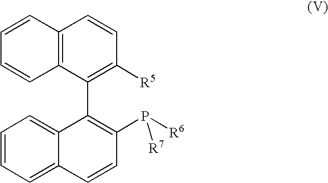
wherein
R⁵ is H; C₁₋₆ alkyl; or —OR⁸, where R⁸ is C₁₋₆ alkyl, C₃₋₇ cycloalkyl, or C₆₋₁₀ aryl; and
R⁶ and R⁷ each are independently C₆₋₁₀ aryl;
wherein each alkyl, cycloalkyl, and aryl is independently, optionally substituted with one or more substituents Q, each of which is independently selected from the group consisting of cyano, halo, or nitro; C₁₋₆ alkyl, C₂₋₆ alkenyl, C₂₋₆ alkynyl, C₃₋₇ cycloalkyl, C₆₋₁₄ aryl, heteroaryl, or heterocyclyl; or —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, or —S(O)₂R^{e}; wherein each R^{e}, R^{f}, R^{g}, and R^{h} is independently hydrogen; C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} together with the N atom to which they are attached form heterocyclyl.

30. The method of claim 27, wherein chiral monodentate phosphine is in R-configuration.

31. The method of claim 27, wherein chiral monodentate phosphine is in S-configuration.

32. The method of claim 29, wherein R⁵ is —OR⁸, where R⁸ is C₁₋₆ alkyl.

33. The method of claim 32, wherein R⁸ is methyl.

34. The method of claim 29, wherein R⁶ and R⁷ each are independently phenyl, optionally substituted with one or more halo groups.

35. The method of claim 27, wherein the silane is a compound of Formula (IV): 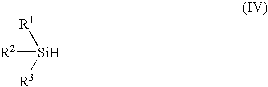
wherein R¹, R², and R³ are each independently H; halogen; C₁₋₆ alkyl; or —OR⁴, where R⁴ is C₁₋₆ alkyl, C₃₋₇ cycloalkyl, or C₆₋₁₀ aryl;
wherein each alkyl, cycloalkyl, and aryl is independently, optionally substituted with one or more substituents Q, each of which is independently selected from the group consisting of cyano, halo, or nitro; C₁₋₆ alkyl, C₂₋₆ alkenyl, C₂₋₆ alkynyl, C₃₋₇ cycloalkyl, C₆₋₁₄ aryl, heteroaryl, or heterocyclyl; or —C(O)R^{e}, —C(O)OR^{e}, —C(O)NR^{f}R^{g}, —C(NR^{e})NR^{f}R^{g}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{f}, —NR^{e}C(O)OR^{f}, —NR^{e}C(O)NR^{f}R^{g}, —NR^{e}C(═NR^{h})NR^{f}R^{g}, —NR^{e}S(O)R^{f}, —NR^{e}S(O)₂R^{f}, —NR^{e}S(O)NR^{f}R^{g}, —NR^{e}S(O)₂NR^{f}R^{g}, —SR^{e}, —S(O)R^{e}, or —S(O)₂1e; wherein each R^{e}, R^{f}, R^{g}, and R^{h} is independently hydrogen; C₁₋₆ alkyl, C₂₋₆ alkenyl, C₂₋₆ alkynyl, C₃₋₇ cycloalkyl, C₆₋₁₄ aryl, heteroaryl, or heterocyclyl; or R^{f} and R^{g} together with the N atom to which they are attached form heterocyclyl.

36. The method of claim 35, wherein the silane is trichlorosilane, methyldichlorosilane, dimethylchlorosilane, methoxydichlorosilane, triethylsilane, pentamethyldisiloxane (HSiMe₂OTMS), or 1,1-dimethyl-3,3-diphenyl-3-tert-butyldisiloxane (HSiMe₂OTBDPS).

37. The method of claim 35, wherein the silane is trichlosilane.

38. The method of claim 27, wherein the transition metal is platinum, iridium, palladium, rhodium, or ruthenium.

39. A method for preparing the compound of claim 1, comprising the steps of:
a) selectively hydrolyzing ester 11 with a hydrolytic enzyme to produce optically active (−)-ester 11 and optically active (+)-alkenol 9;
b) hydrolyzing the optically active (−)-ester 11 to produce optically active (−)-alkenol 9;
c) reducing the optically active (−)-alkenol 12 to produce optically active (−)-alkanol 7; and
d) converting the optically active (−)-alkanol 7 to optically active (+O-exo/C-exo-tricyclo[5.2.1.0^{2,6}]-dec-9-yl-xanthic acid 1A, or a pharmaceutically acceptable salt, solvate, or prodrug thereof.

40. A method for preparing optically active (+)-O-exo/C-exo-tricyclo[5.2.1.0^{2,6}]-dec-9-yl-xanthic acid, or a pharmaceutically acceptable salt, solvate, or prodrug thereof, which comprises the steps of:
a) selectively hydrolyzing ester 11 with a hydrolytic enzyme to produce optically active (−)-ester 11 and optically active (+)-alkenol 9;
b) reducing the optically active (+)-alkenol 12 to produce optically active (+)-alkanol 7; and
c) converting the optically active (+)-alkanol 7 to optically active (+)-β-exo/C-exo-tricyclo[5.2.1.0^{2,6}]-dec-9-yl-xanthic acid 1A. or a pharmaceutically acceptable salt, solvate, or prodrug thereof.

41. The method of claim 39, wherein the hydrolytic enzyme is Rhizopus oryzae peptidase, Candida antactica lipase A, or Pseudomonas fluorescens lipase.

42. The method of claim 41, wherein the hydrolytic enzyme is Rhizopus oryzae peptidase.

43. The method of claim 41, wherein the hydrolytic enzyme is Candida antactica lipase A.

44. The method of claim 41, wherein the hydrolytic enzyme is Pseudomonas fluorescens lipas.

45. A method for preparing the compound of claim 1, comprising the steps of:
a) selectively hydrolyzing ester 13 with a hydrolytic enzyme to produce optically active (−)-ester 13 and optically active (+)-alkanol 7;
b) hydrolyzing the optically active (−)-ester 13 to produce optically active (−)-alkanol 7; and
c) converting the optically active (−)-alkanol 7 to optically active (−)-O-exo/C-exo-tricyclo[5.2.1.0^{2,6}]-dec-9-yl-xanthic acid 1A, or a pharmaceutically acceptable salt, solvate, or prodrug thereof.

46. A method for preparing optically active (+)-O-exo/C-exo-tricyclo[5.2.1.0^{2,6}]-dec-9-yl-xanthic acid, or a pharmaceutically acceptable salt, solvate, or prodrug thereof, which comprises the steps of:
a) selectively hydrolyzing ester 13 with a hydrolytic enzyme to produce optically active (−)-ester 13 and optically active (+)-alkanol 7; and
b) converting the optically active (+)-alkanol 7 to optically active (+)-O-exo/C-exo-tricyclo[5.2.1.0^{2,6}]-dec-9-yl-xanthic acid 1A, or a pharmaceutically acceptable salt, solvate, or prodrug thereof.

47. The method of claim 45, wherein the hydrolytic enzyme is Rhizopus oryzae peptidase, Candida antactica lipase A, or Pseudomonas fluorescens lipase.

48. The method of claim 47, wherein the hydrolytic enzyme is Rhizopus oryzae peptidase.

49. The method of any of claims 39, wherein the hydrolytic enzyme is in a catalytic amount.

50. A method for treating, preventing, or ameliorating a disease selected from the group consisting of cancer, autoimmune diseases, inflammatory diseases, neurodegenerative diseases, and diseases associated with ischemia, reperfusion injury, trauma, atherosclerosis, and/or aging; which comprises administering to a subject the compound of claim 1.

51. The method of claim 50, wherein the cancer is breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, leukemia, stomach cancer, pancreatic cancer, testicular lymphoma, or multiple myeloma.
